SEQUENCE LISTING (30 sequences)

SEQ ID NO: 1 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 2 - protein, 23 aa, Artificial Sequence
  synthetic
METDTLLLWVLLLWVPGSTGAAS

SEQ ID NO: 3 - DNA, 723 bp, Homo sapiens
atggagacagacacactcctgctatgggtactgctgctctgggttccaggttccactggcgccgctagcgatattcaaatgacccaatccccgagcagcctgagtgccagcgtaggagatcgggttacgataacgtgtcgagcatccgaaaatatctatagctatgtagcatggtaccaacaaaaacctggcaaggcaccgaaactgttgatatacaacgccaagagtctggcaagtggggtgccttcacgcttcagtggaagcggaagtgggacagattttacacttaccatttcctcccttcagcctgaagactttgcaacatattattgtcaacatcactatgtgagtccgtggactttcgggggaggaaccaaattggaaataaagcgcacagtcgcagcgcccagtgtgtttatattccccccttcagacgaacagctcaaatccggcacagcatcagtggtgtgtctgttgaacaatttctatcctagagaggcaaaggttcaatggaaagtggacaacgcgctccaaagcgggaactcccaagagagcgtcactgaacaagattccaaagatagcacgtactctctttcttcaacgctcacgctcagtaaggccgattacgagaaacataaggtatacgcttgcgaggtcacgcatcaagggctgtcctcacccgtgaccaagtctttcaaccggggggaatgttaataaatgcat

SEQ ID NO: 4 - protein, 108 aa, Homo sapiens
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKR

SEQ ID NO: 5 - protein, 214 aa, Homo sapiens
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 720 bp, Homo sapiens
atggagacagacacactcctgctatgggtactgctgctctgggttccaggttccactggcgccgctagccaggccgtagtgacacaggaaccgtctttgacggtgtctccgggaggtaccgtcaccttgacgtgtgggtccagcactggagctgtaacaacgagcaattacgcgaattgggtgcaggagaagccaggtcaggcttttaggggtcttatcggagggactaataaaagggctccaggcacgccggcaagattctcagggtccctgctgggggggaaagcggcactcaccctttctggtgctcagccagaggatgaggccgaatattattgtgccttgtggtattctaatttgtgggtctttggaggcgggacaaaactcactgtattgtcatctgcgtcaacgaagggaccttctgtattccccttggcaccatccagtaaatctaccagtgggggtaccgctgccctcggttgccttgtaaaagattactttccggagcccgtcaccgtgtcctggaacagcggggcattgaccagtggtgtccacacttttcccgcagtactccaaagctccggcctctacagtctctcttcagttgtgacggttcctagctcttcccttggtacgcagacttatatctgcaacgtcaaccacaaaccttccaatactaaggtagacaaaaaggtggagcccaaatcttgttaaatgcat

SEQ ID NO: 7 - protein, 109 aa, Homo sapiens
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 8 - protein, 214 aa, Homo sapiens
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - DNA, 2214 bp, Homo sapiens
atggagacagacacactcctgctatgggtactgctgctctgggttccaggttccactggcgccgctagccaggtacaattgcaggaatcaggacccggattggtgaagccaagtgaaactctgagtttgacttgcacagtcagcggcttctcactgacatcctatggggtaagttggattcggcaaccggcaggtaagggccttgaatggatcggggtcatctgggaagatgggtcaactaactaccattctgcgctgataagtcgcgttaccatgtcagtcgatacaagcaagaatcagttctctctgaaactgagcagtgtgacagccgcagacaccgcagtgtactattgcgcacgccctcactacggatcctcttatgtgggagcaatggaatattggggagcgggaacaacagtaacagtttcttcagcgagcactaagggcccgtctgtatttccccttgccccttcatccaagagcacgagtggtggaacggccgcactcggatgtttggtaaaagactatttcccagagcccgtgactgtgtcttggaattccggtgcactgacttctggtgtgcatacttttccggccgtgcttcaaagttcaggcctttatagcttgagctcagtagtcaccgtcccttcatcttcattggggacacaaacctatatctgtaatgttaatcataaaccttccaacaccaaagtcgacaagaaagtggagccaaagagttgcgatgggggagggggaagcgggggagggggttcagaggtacagctgctcgaaagcggcggaggtcttgtgcaaccaggcgggtcactcagactcagttgtgcggcgtccggcttcacttttagtacgtatgccatgaactgggtgcgacaagctcctggcaagggactggagtgggtgtcccggataagatcaaaatataacaattacgcgacctattatgcagatagtgtcaaaggaaggtttacaatatctcgcgatgattctaaaaatacgctctatctgcagatgaatagtttgcgggccgaagataccgcggtatattattgtgtccgccacgggaattttggcaactcttacgttagttggttcgcttattgggggcagggaactctggtcacagtatccagtgcatcagttgcggccccctcagtcttcatttttccccctagtgatgagcaacttaagtccggaacagccagcgtggtctgcctgctgaacaatttttatccgagggaggcgaaggttcaatggaaagtcgataacgctctgcaatcaggtaattctcaggaatctgtcactgaacaagatagtaaagacagcacatactctttgtcttctacattgaccttgtctaaggcggattacgaaaagcataaggtctatgcttgcgaagtgacgcatcaggggcttagttccccggtcaccaagagtttcaataggggggagtgcgataagacccacacctgtccgccatgccctgcacctgaggcagcgggagggccgagtgtattcttgttccctccaaaaccgaaagatactctgatgattagccggacccccgaagttacgtgtgtggttgtagacgtaagtcacgaagatcctgaagttaagtttaactggtatgttgatggggtggaagttcacaatgccaaaaccaaacctagagaggagcaatacaactccacctatcgggttgtaagcgtcttgaccgtgctccaccaagactggctgaacggtaaggagtataagtgtaaggtgagcaacaaggctttgggagcacccatcgaaaaaacgatcagcaaagccaaaggtcagccacgcgaaccccaggtgtatacccttccgccttgtagggatgagcttactaagaaccaagtttcactctggtgtctggtgaagggtttttacccctccgatattgctgtggagtgggagtcaaacgggcagccagaaaataactataagaccacgccacctgtccttgacagtgacggaagttttttcctgtattctaaattgaccgtagataagtctcgatggcagcaaggaaacgtgttttcatgctctgttatgcacgaagctctccacaaccattatacacaaaagtcactgagccttagtcctggtaaaatgcatgaggctctgcacaaccactacacgcagaagagcctctccctgtctcccgggaaatga

SEQ ID NO: 10 - protein, 121 aa, Homo sapiens
QVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSS

SEQ ID NO: 11 - protein, 125 aa, Homo sapiens
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 12 - protein, 714 aa, Homo sapiens
QVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKMHEALHNHYTQKSLSLSPGK

SEQ ID NO: 13 - DNA, 1479 bp, Homo sapiens
atggagacagacacactcctgctatgggtactgctgctctgggttccaggttccactggcgccgctagccaggtccagctgcaagagtccggacctgggctggtcaaacccagtgagacgttgtcccttacgtgtactgtctccggcttcagtttgacgtcttatggagtttcttggatacggcagcccgccggtaagggcctcgagtggattggagttatatgggaagatgggtccactaattatcatagcgcccttattagcagggtaaccatgtctgtcgatactagcaaaaatcagttcagccttaaattgtcaagtgtgaccgctgcagatacagcagtatattactgtgcgagaccacattatggatccagttatgtcggagcgatggagtattggggcgcgggcaccactgttacggttagcagcacaaagggaccgtctgtgttcccccttgcgccgtcttccaagtccactagtggggggaccgcggcactgggatgcctggttaaggactacttcccggaaccggtgaccgttagttggaacagtggcgcccttacgtcaggggttcatacgtttcccgcggtgctgcaaagcagtggactctatagtctttcttcagtagttacagtgcctagcagcagtctcggcacgcagacatatatatgtaatgttaatcataaaccctcaaatacaaaggtagacaagaaagtagagccaaagagttgcgacaagacacatacttgccctccgtgccctgcacccgaagcggcaggcggcccatcagtatttttgtttcctcctaaacctaaagacactcttatgatatcacggacacctgaagtcacttgtgtagttgtggacgtttcacatgaggatcccgaagtcaagttcaactggtacgtcgatggcgtagaagttcataacgcaaaaacaaagccgcgggaggagcagtataactcaacctatcgagtagtctctgttcttacggttttgcatcaagactggctcaatggtaaggagtataaatgtaaagtgagcaataaagctctgggtgctcctatagagaaaacgatctctaaggcgaaagggcaacctcgggagccgcaggtgtgtacattgccccccagccgagatgaactcactaaaaaccaagtttccctcagctgcgcagtcaagggattctacccgagcgacattgcagtagagtgggagtcaaatggtcagccagaaaataattataaaacaacccctcccgtcctggacagcgatggatcattctttctggtgtctaaacttacagtggataaatcccgatggcaacaaggcaatgtgttttcttgttctgtaatgcacgaggcattgcataatcactacactcagaaatctctcagccttagtccagggaaaatgcatgaggctctgcacaaccactacacgcagaagagcctctccctgtctcccgggaaatga

SEQ ID NO: 14 - protein, 469 aa, Homo sapiens
QVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKMHEALHNHYTQKSLSLSPGK

SEQ ID NO: 15 - DNA, 718 bp, Homo sapiens
atggagacagacacactcctgctatgggtactgctgctctgggttccaggttccactggcgccgctagcgacatacagatgactcaaagcccctcttcactgtctgcatcagtcggggacagagtcacaataacctgcagagcgagcgagaatatctactcttatgtagcctggtatcagcaaaaacccggcaaggcgccgaaattgctcatctataatgcgaaatccttggccagtggggtcccatcacggttcagtggctccggctctggaaccgatttcacactcacaatctctagcctccagcccgaagacttcgccacatactattgccaacatcactatgtcagcccatggacatttgggggaggtacgaaacttgaaattaaacgtacagtagctgctccgtccgtctttattttcccgccgtctgacgaacagctcaaaagcgggactgcatcagttgtctgtctcctcaacaatttttacccgcgagagaacttgttcaatggaaagttgataacgccctccagagtggaaactctcaggagagtgtaactgagcaagattccaaagattcaacctatagtctttcaagtaccttgactctttctaaagcggattatgagaaacataaagtgtatgcctgcgaagtgacccatcaggggctttcatcacccgtgacgaagtccaacttcgaggcgaatgctaaatgcat

SEQ ID NO: 16 - protein, 108 aa, Homo sapiens; identical to SEQ ID NO: 4

SEQ ID NO: 17 - protein, 214 aa, Homo sapiens; identical to SEQ ID NO: 5

SEQ ID NO: 18 - DNA, 2148 bp, Homo sapiens
atggagacagacacactcctgctatgggtactgctgctctgggttccaggttccactggcgccgctagccaggttcaacttcaagaatcaggaccgggcttggttaaaccttccgaaactctgagccttacttgtacagtgtctggtggatctattacgagctacggagtaagttggatccggcaaccacccgggaaagggctcgaatggataggggtgatatgggaggatggttcaaccaactaccatagcgctctgatcagccgggtgaccattagtgtcgacacttccaaaaaccagttttcattgaagctctcaagcgtaactgcggcggataccgccgtatactattgtgcgcggccacattacgggtcctcttatgttggggcgatggaatattggggggcaggtacaacggtcacggtgtcttcaacaaaaggtccttccgtatttccgctcgcacccagctctaagtcaacctctggcggtactgcagccctgggttgcctcgtaaaggactattttcctgagccagtaacagtttcttggaacagcggggcacttacgagcggtgttcatacgttccctgcagtgttgcaatccagcggcctttattcattgtcttcagttgtaacggttccttctagtagtttggggacccagacatatatctgcaacgtgaaccataagccaagcaataccaaagttgataagaaggtcgaacctaagtcctgcgacggcgggggaggatctggcgggggaggcagtgaggtacaattgttggagtcaggcggcggtcttgtccaaccgggagggtccctgagactgtcctgtgcggccagcgggtttactttttcaacatatgccatgaactgggttcggcaagcaccaggtaagggactggaatgggttagtcgaattaggtccaagtataataattacgcaacctactacgctgactctgtcaaggggcggtttaccatatctagggatgactccaaaaacacattgtacttgcaaatgaacagcctgagggcagaagacaccgcagtctactattgtgtacgccacggaaacttcgggaatagttatgtctcctggttcgcttactggggtcagggaacactggtcacagtctcatcagccagtgtagcggccccgtccgttttcatattccctccttccgacgagcagttgaaaagcggtacggcgagcgttgtgtgcttgttgaacaacttctacccacgcgaagccaaggtccaatggaaggtagacaacgcactgcagagtggtaactcacaggaatcagtgacggaacaggactcaaaagatagtacttacagtctttcttccacactgacactcagtaaggccgattatgagaaacataaagtatacgcatgtgaagtaactcaccagggtctcagttcaccagtaactaagtctttcaatcgcggggaatgcgacaaaacacacacctgtcccccctgtccagccccagaggcagctggcggccctagtgtgttcttgttcccgcccaagccaaaagatacactgatgattagccggacccctgaggtaacttgtgtggtggtggacgtgtctcatgaggacccagaggtaaaattcaactggtacgtagacggcgtcgaggtccataatgccaaaaccaagccacgggaggagcagtataattccacttatcgcgtagtctctgtacttacagttcttcaccaagattggttgaacggaaaagaatacaagtgtaaagttagcaataaggcgctcggagctccgatcgaaaaaacaatctccaaagcaaaagggcaaccccgagaaccacaggtatacaccctgccgccgtgccgagacgagctgacgaaaaaccaagtgtccctgtggtgcttggtgaagggcttttatccaagtgacattgcagttgaatgggagtctaacggacagcctgaaaataactataagaccacgccaccagtccttgatagcgatggatctttttttctctatagcaagttgactgtagataaatcacgatggcaacaaggcaatgtcttttcatgcagcgttatgcatgaggctctgcacaaccactacacgcagaagagcctctccctgtctcccgggaaatga

SEQ ID NO: 19 - protein, 121 aa, Homo sapiens
QVQLQESGPGLVKPSETLSLTCTVSGGSITSYGVSWIRQPPGKGLEWIGVIWEDGSTNYHSALISRVTISVDTSKNQFSLKLSSVTAADTAVYYCARPHYGSSYVGAMEYWGAGTTVTVSS

SEQ ID NO: 20 - protein, 692 aa, Homo sapiens
QVQLQESGPGLVKPSETLSLTCTVSGGSITSYGVSWIRQPPGKGLEWIGVIWEDGSTNYHSALISRVTISVDTSKNQFSLKLSSVTAADTAVYYCARPHYGSSYVGAMEYWGAGTTVTVSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1419 bp, Homo sapiens
atggagacagacacactcctgctatgggtactgctgctctgggttccaggttccactggcgccgctagccaggttcaacttcaagaatcaggaccgggcttggttaaaccttccgaaactctgagccttacttgtacagtgtctggtggatctattacgagctacggagtaagttggatccggcaaccacccgggaaagggctcgaatggataggggtgatatgggaggatggttcaaccaactaccatagcgctctgatcagccgggtgaccattagtgtcgacacttccaaaaaccagttttcattgaagctctcaagcgtaactgcggcggataccgccgtatactattgtgcgcggccacattacgggtcctcttatgttggggcgatggaatattggggggcaggtacaacggtcacggtgtcttcaaccaaaggtccaagcgtatttccactcgcaccgtcctccaaatcaacgagtggaggtactgcggcgttgggatgcttggtgaaagactatttcccagagccggtgacagttagttggaactcaggcgcgctgactagtggtgttcacacatttcctgcagttttgcaatcctcaggcctctatagcctgtcaagcgtcgtgacagtacctagcagctcacttggcacgcagacatatatttgtaatgtcaaccataagccctctaacacaaaggtagataagaaggttgaacccaagtcctgtgacaaaacgcacacttgtccaccatgtccagcgcccgaagcagccgggggcccaagcgtgttcctcttccctcccaagccaaaagacacccttatgatctcaaggactccagaagtgacatgcgtagtcgttgacgtaagtcacgaggatccggaagtgaagttcaactggtacgtggacggtgtggaggtacataacgcgaagactaagcccagagaagaacaatataactcaacctaccgggtcgtttctgtgctcacagtgctccaccaggactggcttaacggaaaagagtataaatgcaaagtatctaataaagcgctcggagcgcccatagagaaaactatttctaaagcaaaaggtcaaccacgggagccgcaggtttgcacacttccaccgtccagggatgaacttactaagaaccaggtatctctttcttgtgccgtgaaaggtttttatcctagtgacatcgctgtcgagtgggagagcaacggtcagccggagaataactataagaccacacctccggttctggattctgacggctctttcttcctggtatctaagcttacagtcgataaaagtcgatggcaacaagggaatgtttttagctgctctgtgatgcatgaggctctgcacaaccactacacgcagaagagcctctccctgtctcccgggaaatga

SEQ ID NO: 22 - protein, 449 aa, Homo sapiens
QVQLQESGPGLVKPSETLSLTCTVSGGSITSYGVSWIRQPPGKGLEWIGVIWEDGSTNYHSALISRVTISVDTSKNQFSLKLSSVTAADTAVYYCARPHYGSSYVGAMEYWGAGTTVTVSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - DNA, 2211 bp, Homo sapiens
atggagacagacacactcctgctatgggtactgctgctctgggttccaggttccactggcgccgctagccaggttcaacttcaagaatcaggaccgggcttggttaaaccttccgaaactctgagccttacttgtacagtgtctggtggatctattacgagctacggagtaagttggatccggcaaccacccgggaaagggctcgaatggataggggtgatatgggaggatggttcaaccaactaccatagcgctctgatcagccgggtgaccattagtgtcgacacttccaaaaaccagttttcattgaagctctcaagcgtaactgcggcggataccgccgtatactattgtgcgcggccacattacgggtcctcttatgttggggcgatggaatattggggggcaggtacaacggtcacggtgtcttcaacaaaaggtccttccgtatttccgctcgcacccagctctaagtcaacctctggcggtactgcagccctgggttgcctcgtaaaggactattttcctgagccagtaacagtttcttggaacagcggggcacttacgagcggtgttcatacgttccctgcagtgttgcaatccagcggcctttattcattgtcttcagttgtaacggttccttctagtagtttggggacccagacatatatctgcaacgtgaaccataagccaagcaataccaaagttgataagaaggtcgaacctaagtcctgcgacaaaacacacacctgtcccccctgtccagccccagaggcagctggcggccctagtgtgttcttgttcccgcccaagccaaaagatacactgatgattagccggacccctgaggtaacttgtgtggtggtggacgtgtctcatgaggacccagaggtaaaattcaactggtacgtagacggcgtcgaggtccataatgccaaaaccaagccacgggaggagcagtataattccacttatcgcgtagtctctgtacttacagttcttcaccaagattggttgaacggaaaagaatacaagtgtaaagttagcaataaggcgctcggagctccgatcgaaaaaacaatctccaaagcaaaagggcaaccccgagaaccacaggtatacaccctgccgccgtgccgagacgagctgacgaaaaaccaagtgtccctgtggtgcttggtgaagggcttttatccaagtgacattgcagttgaatgggagtctaacggacagcctgaaaataactataagaccacgccaccagtccttgatagcgatggatctttttttctctatagcaagttgactgtagataaatcacgatggcaacaaggcaatgtcttttcatgcagcgttatgcatgaggctctgcacaaccactacacgcagaagagcctctccctgtctcccgggaaaggcgggggaggatctggcgggggaggcagtggcgggggaggatctgaggtacaattgttggagtcaggcggcggtcttgtccaaccgggagggtccctgagactgtcctgtgcggccagcgggtttactttttcaacatatgccatgaactgggttcggcaagcaccaggtaagggactggaatgggttagtcgaattaggtccaagtataataattacgcaacctactacgctgactctgtcaaggggcggtttaccatatctagggatgactccaaaaacacattgtacttgcaaatgaacagcctgagggcagaagacaccgcagtctactattgtgtacgccacggaaacttcgggaatagttatgtctcctggttcgcttactggggtcagggaacactggtcacagtctcatcaggcgggggaggatctggcgggggaggcagtggcgggggaggatctcaggccgtagtgacacaggaaccgtctttgacggtgtctccgggaggtaccgtcaccttgacgtgtgggtccagcactggagctgtaacaacgagcaattacgcgaattgggtgcaggagaagccaggtcaggcttttaggggtcttatcggagggactaataaaagggctccaggcacgccggcaagattctcagggtccctgctgggggggaaagcggcactcaccctttctggtgctcagccagaggatgaggccgaatattattgtgccttgtggtattctaatttgtgggtctttggaggcgggacaaaactcactgtattgtaa

SEQ ID NO: 24 - protein, 713 aa, Homo sapiens
QVQLQESGPGLVKPSETLSLTCTVSGGSITSYGVSWIRQPPGKGLEWIGVIWEDGSTNYHSALISRVTISVDTSKNQFSLKLSSVTAADTAVYYCARPHYGSSYVGAMEYWGAGTTVTVSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 25 - protein, 249 aa, Homo sapiens
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 26 - protein, 20 aa, Artificial Sequence
  synthetic
METDTLLLWVLLLWVPGSTG

SEQ ID NO: 27 - DNA, 1440 bp, Homo sapiens
atggagacagacacactcctgctatgggtactgctgctctgggttccagggtcgactggcgaagtgcagcaggtgcagcttcaggaaagtggaccgggccttgtcaaaccgtcagagaccctttcactgacttgcactgtaagtggtttctccctgacaagctacggagtctcctggatacgccagccagcggggaaagggcttgagtggatcggtgtgatctgggaagacgggagtacaaactatcactcagcactcattagtcgagtaacaatgtccgttgacacttccaagaatcaattcagtttgaaactgtctagtgtgacggctgcggatacagcggtttattactgtgccaggcctcattacggaagttcttatgttggtgcaatggagtattggggagccggcacaactgtcactgtgagctccgtcaccgtctcaagcgcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctccggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 28 - protein, 456 aa, Homo sapiens
QVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1509 bp, Homo sapiens
atggagacagacacactcctgctatgggtactgctgctctgggttccagggtcgactggcgaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacatatcctactgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactatccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctaggcggcggaggaagtgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 30 - protein, 482 aa, Homo sapiens
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSAAATHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2022/0348688 A1
(43) Pub. Date: Nov. 3, 2022

(54) PLAP-CD3 EPSILON BISPECIFIC ANTIBODIES

(71) Applicants: ProMab Biotechnologies, Inc., Richmond, CA (US); Forevertek Biotechnology Co., Ltd, Changsha (CN)

(72) Inventors: Lijun Wu, Albany, CA (US); Vita Golubovskaya, Richmond, CA (US)

(21) Appl. No.: 17/809,699

(22) Filed: Jun. 29, 2022

Related U.S. Application Data
(63) Continuation of application No. PCT/US2021/013916, filed on Jan. 19, 2021.
(60) Provisional application No. 62/966,846, filed on Jan. 28, 2020.

(51) Int. Cl. C07K 16/40; C07K 16/28

(52) U.S. Cl. CPC C07K 16/40; C07K 16/2809; A61K 2039/505

(57) ABSTRACT

The present invention is directed to bispecific humanized PLAP (placental alkaline phosphatase)-CD3 epsilon chain (CD3e) antibodies. The present invention is further directed to a method for treating PLAP-positive cancer cells by administering the bispecific PLAP-CD3e antibody to the patients.

This application is a continuation of PCT/US2021/013916, filed Jan. 19, 2021; which claims the priority of U.S. Provisional Application No. 62/966,846, filed Jan. 28, 2020. The contents of the above-identified applications are incorporated herein by reference in their entireties.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence Listing.txt with a creation date of Jan. 14, 2021, and a size of 72.1 kilobytes. The Sequence Listing filed via EFS-Web is part of the specification and is hereby incorporated in its entirety by reference herein.

### FIELD OF THE INVENTION

The present invention relates to PLAP (placental alkaline phosphatase)-CD3 epsilon chain (CD3e) bispecific antibodies. The present invention is also directed to a method for killing PLAP-positive cancer cells by administering PLAP-CD3e bispecific antibody with T cells to the patients.

### BACKGROUND OF THE INVENTION

Immunotherapy is emerging as a highly promising approach for the treatment of cancer. T cells or T lymphocytes, the armed forces of our immune system, constantly look for foreign antigens and discriminate abnormal (cancer or infected cells) from normal cells. Using bispecific antibodies binding T cells and tumor associated antigen is the most common approach to design bispecific antibody by bringing cytotoxic T cells to kill cancer cells. Bispecific antibodies can be infused into patients by different routes. The advantage of bispecific antibodies compared with chemotherapy or antibody is that it specifically targets antigen-positive cancer cells and simultaneously activates T cells.

Redirecting the activity of T cells by bispecific antibodies against tumor cells, independently of their TCR specificity, is a potent approach to treat cancer. The concept is based on recognition of a cell surface tumor antigen and simultaneous binding to the CD3 epsilon chain (CD3e) within the T-cell receptor (TCR) complex on T cells. This triggers T-cell activation, including release of cytotoxic molecules, cytokines and chemokines, and induction of T-cell proliferation.

### PLAP

PLAP is a placental alkaline phosphatase that is encoded by ALPP gene. PLAP is a metalloenzyme enzyme that catalyzes the hydrolysis of phosphoric acid monoesters. PLAP is expressed mainly in placental and endometrial tissues, it is not expressed in normal tissues. PLAP has high expression in placenta (1), and it is not expressed in most normal tissues except of testis (2). It was found to be overexpressed in malignant seminoma, teratoma (2), (3), ovarian and cervical carcinoma (3), (4),(5), and colon adenocarcinoma (6). PLAP was detected in lung, pancreas, stomach tumors (7). PLAP was also detected among several other membrane-bound proteins in exosomes of non-small cell lung cancer patients with a potential to be prognostic marker (8).

Human PLAP is a 535 amino-acid glycosylated protein encoded by ALPP gene with 1-22 signaling peptide, then extracellular domain (23-506), 513-529 transmembrane domain (sequence is shown below, transmembrane domain is underlined) Uniprot database (www.uniprot.org/uniprot/P05187; NM_001632). Its sequence is shown below (SEQ ID NO: 1).

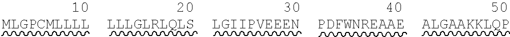

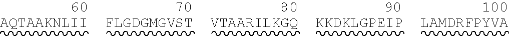

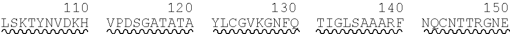

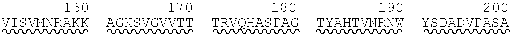

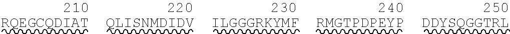

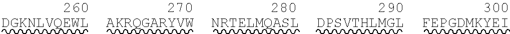

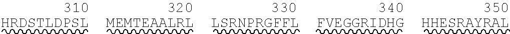

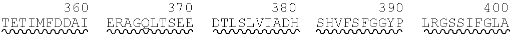

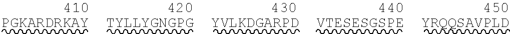

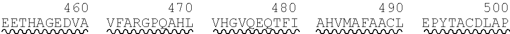

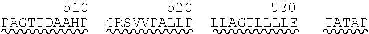

There are four distinct but related alkaline phosphatases: intestinal (encoded by ALPI) (NM_001631); placental (ALPP); placental-like (ALPPL2) (NM_031313) which are all encoded by gene on at chromosome 2 and liver/bone/kidney (ALPL) (tissue-nonspecific) (NM_000478) encoded by gene on chromosome 1.

### BRIEF DESCRIPTION OF THE DRAWINGS

FIGS. 1A-1C show the structures of bi-specific humanized PLAP and CD3 antibodies. FIG. 1A shows # 1-4 DNA constructs encoding four polypeptides. FIG. 1B shows # 1-3 DNA constructs encoding 3 polypeptides of bivalent PLAP-CD3 antibody. FIG. 1C shows # 1-3 DNA constructs encoding 3 polypeptides of humanized univalent PLAP-CD3 antibody. The antibody of FIGS. 1A and 1B have two PLAP binding moieties and one CD3 binding moiety. The antibody of FIG. 1C has one PLAP binding moiety and one CD3 binding moiety. The knobs-in-hole structure and silent Fc mutations P329G and leucine to alanine (L234A, L235A or LA-LA) mutations are shown in structures FIGS. 1A and 1B; and LA-LA only for FIG. 1C. The amino acid numbers in CH3 are counted from human IgG1 according to [10].

FIG. 2 shows expression of PLAP-h2-CD3 and PLAP-h4-CD3 antibodies on SDS gel. The supernatant shows higher 206 kDa band at non-reducing conditions (B) and lower molecular bands at reducing conditions (C). A shows molecular weight marker (KDa) with proteins marked in kDa.

FIG. 3 shows purification of PLAP-h2-CD3 antibody. PLAP h2 (chimeric form was used with Fc nucleotide sequence with different codon optimization)-CD3 antibody. A-non-reduced; B-reduced conditions; C-molecular marker, molecular weight is shown in kDa.

FIG. 4 shows binding of PLAP-CD3 antibody with CD3 and PLAP antigens by FACS. Bispecific antibodies used with PLAP-positive and PLAP-negative cell lines. CD3-positive T cells were used for testing binding. Bispecific antibodies had positive binding with both PLAP and Cd3 antigens. PLAP h2 -CD3 antibody is shown, the same was observed for PLAP h4-CD3 antibody (not shown).

FIGS. 5A-5B show real-time cytotoxicity assay. PLAP h2-CD3 bispecific antibody with T cells killed Lovo (PLAP-positive) cells and did not kill HT29 (PLAP-negative) cells. T cells ratio to target cells was 5:1 (E:T).

FIGS. 6A-6B show real-time cytotoxicity assay. PLAP h4-CD3 antibody with T cells killed Lovo (PLAP-positive) cells and did not kill PLAP-negative cells. T cells were used at E:T ratio 5:1 (T to target cells)

FIG. 7 shows that PLAP h2-CD3 antibody plus T cells significantly decreased Lovo xenograft tumor growth. P=0.007 at day 18 versus Mock T cells, Student's t-test.

FIG. 8 shows that bivalent PLAP h4-CD3 Ab PBM0015 (FIG. 1B structure) runs as a single band on SDS gel with Molecular Weight 130 kDa.

FIG. 9 shows that bivalent PLAP h4-CD3 (PBM0015) antibody with T cells caused dose-dependent killing of PLAP-positive cells,

FIG. 10 shows that bivalent humanized PLAPh4-CD3 antibody (PBM0015) with T cells secreted significant level of IFN-gamma with Lovo cells but not with HCT116 cells. Concentration of Ab is expressed in ng/ml.

FIGS. 11A-11D show that univalent PLAP h2-3 (PBM008, FIG. 1C structure) with T cells specifically killed PLAP-positive Lovo cells and secrete IFN-gamma. FIGS. 11A-11B: RTCA was performed with PLAP h2-3 and compared with PLAP h2 and PLAP h4 (FIG. 1A structure). PLAPh2-3 had similar e high activity in Lovo cells and low activity in PLAP-negative cells. FIGS. 11C-1D: PLAP h2-3 had high secretion of IFN-gamma with PLAP-positive Lovo target cells, but not with PLAP-negative HCT116 cells.

### DETAILED DESCRIPTION OF THE INVENTION

### Definitions

As used herein, “affinity” is the strength of binding of a single molecule to its ligand. Affinity is typically measured and reported by the equilibrium dissociation constant (KD or Kd), which is used to evaluate and rank order strengths of bimolecular interactions.

As used herein, “bispecific antibody” is an artificial protein that can simultaneously bind to two different types of antigen or different epitopes of the same antigen.

As used herein, “CD3 epsilon (CD3e)” is a polypeptide encoded by the CD3E gene which resides on chromosome 11 in human. CD3-epsilon polypeptide, which together with CD3-gamma, -delta and -zeta, and the T-cell receptor alpha/beta and gamma/delta heterodimers, forms the T cell receptor-CD3 complex. This complex plays an important role in coupling antigen recognition to several intracellular signal-transduction pathways. The CD3 epsilon polypeptide plays an essential role in T-cell development. CD3 epsilon, CD3e, and CD3 are used interchangeably in this application.

As used herein, a “domain” means one region in a polypeptide which is folded into a particular structure independently of other regions.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for preparing an scFv are known to a person skilled in the art.

As used herein, a “tumor antigen” means a biological molecule having antigenicity, expression of which causes cancer.

The inventors have discovered that human PLAP is a unique tumor marker. Unlike other tumor markers that are expressed in low levels in normal tissues, human PLAP is not expressed in most normal tissues but only in placenta and testis. Therefore, PLAP-CD3e bispecific antibodies do not react against normal tissues and they are safe and have low toxicity.

The present invention is directed to bispecific antibodies that specifically binds to both human PLAP and human CD3e. The PLAP-CD3e bispecific antibody targets PLAP tumor antigen which is highly overexpressed in many types of cancer such as ovarian, seminoma, and colon cancer. The PLAP-CD3 bispecific antibodies of the present invention have high cytotoxic activity against several colon cancer cell lines. The bispecific antibody activates T cells and re-directs T cells to PLAP-positive cancer cells.

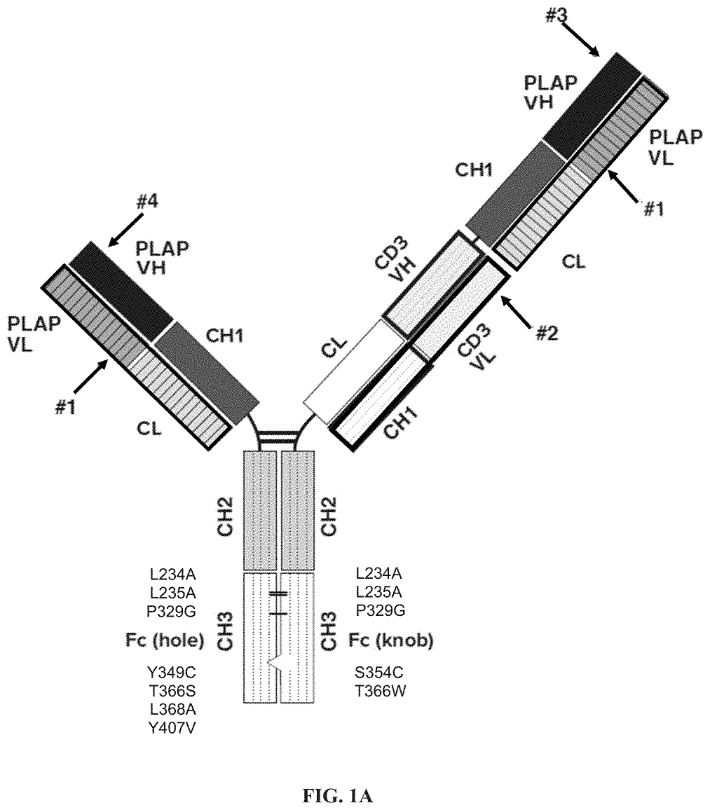

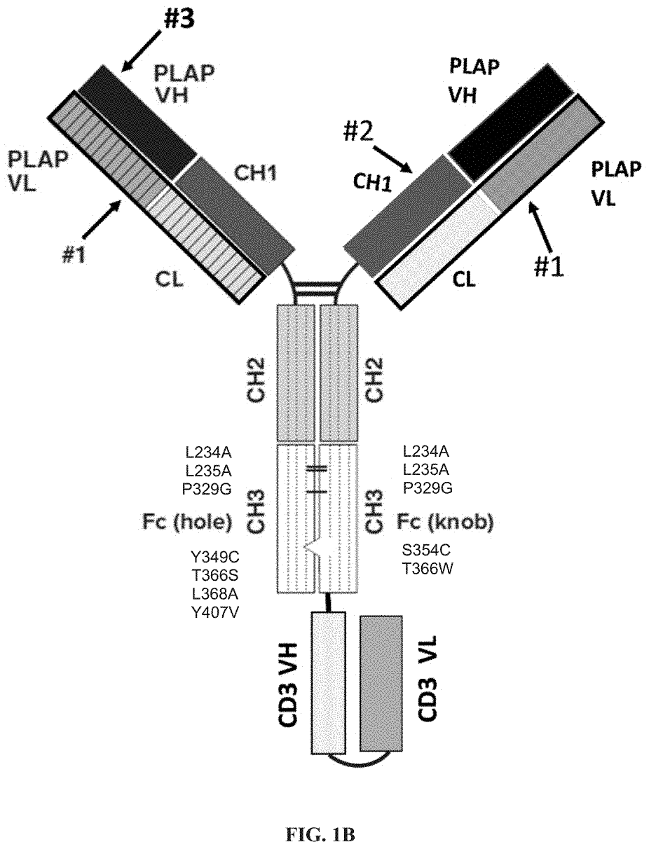

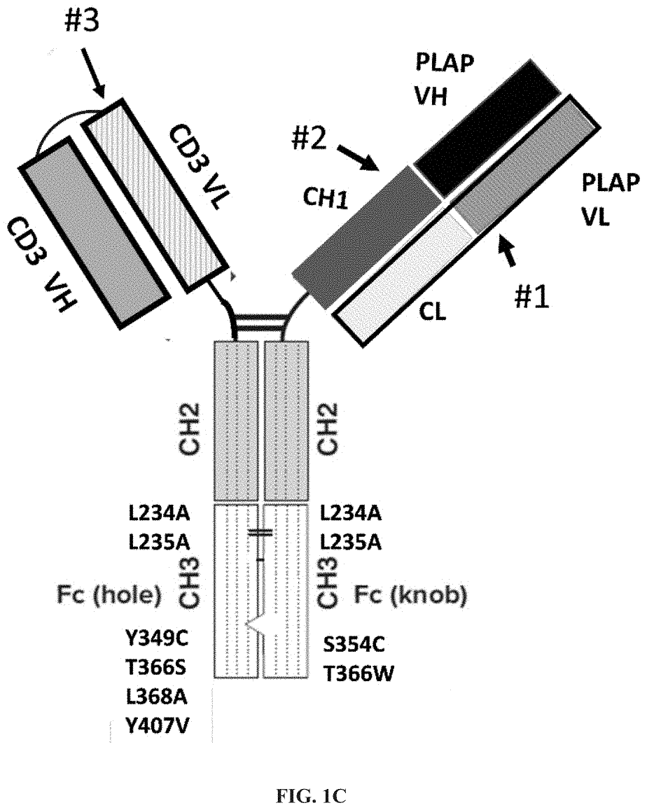

Three bispecific antibody structures of the present invention are shown in FIGS. 1A-1C. FIGS. 1A and 1B shows a heterodimeric antibody that binds with one arm to human CD3e chain expressed on T cells and with two arms to human PLAP expressed on PLAP-positive cancer cells. FIG. 1C shows a heterodimeric antibody that binds with one arm to human CD3e chain and one arm to human PLAP.

### Bispecific Antibody Structure of FIG. 1A

The present invention is directed to a bispecific antigen-binding molecule having structure of FIG. 1A. In one aspect, the PLAP antibody is humanized h2, and the bispecific antibody comprises: (a) a first and a second antigen-binding moiety each of which is a humanized Fab molecule capable of specific binding to human PLAP, and each comprises a heavy chain variable region (PALP VH) having the amino acid sequence of SEQ ID NO: 10 and a light chain variable region (PLAP VL) having the amino acid sequence of SEQ ID NO: 4; (b) a third antigen-binding moiety which is a Fab molecule capable of specific binding to human CD3 epsilon, the third antigen-binding moiety comprises a heavy chain variable region (CD3 VH) having the amino acid sequence of SEQ ID NO: 11 and a light chain variable region (CD3 VL) having the amino acid sequence of SEQ ID NO: 7, wherein the third antigen-binding moiety is a crossover Fab molecule, in which the constant regions of the Fab light chain and the Fab heavy chain are exchanged; and (c) an human IgG Fc domain comprising a first subunit and a second subunit capable of stable association; wherein the Fab heavy chain of the third antigen-binding moiety is (i) fused at the N-terminus to the C-terminus of the Fab heavy chain of the first antigen-binding moiety (CH1), and (ii) fused at the C-terminus to the N-terminus of the first subunit of the Fc knob domain, and wherein the second antigen-binding moiety is fused at the C-terminus of the Fab heavy chain (CH1) to the N-terminus of the second subunit of the Fc hole domain.

In another aspect, the PLAP antibody is humanized h4, and the bispecific antibody comprises: (a) a first and a second antigen-binding moiety each of which is a humanized Fab molecule capable of specific binding to human PLAP, and each comprises a heavy chain variable region (PALP VH) having the amino acid sequence of SEQ ID NO: 19 and a light chain variable region (PLAP VL) having the amino acid sequence of SEQ ID NO: 16; (b) a third antigen-binding moiety which is a Fab molecule capable of specific binding to human CD3 epsilon, the third antigen-binding moiety comprises a heavy chain variable region (CD3 VH) having the amino acid sequence of SEQ ID NO: 11 and a light chain variable region (CD3 VL) having the amino acid sequence of SEQ ID NO: 7, wherein the third antigen-binding moiety is a crossover Fab molecule, in which the constant regions of the Fab light chain and the Fab heavy chain are exchanged; and (c) an human IgG Fc domain comprising a first subunit and a second subunit capable of stable association; wherein the Fab heavy chain of the third antigen-binding moiety is (i) fused at the N-terminus to the C-terminus of the Fab heavy chain of the first antigen-binding moiety (CH1), and (ii) fused at the C-terminus to the N-terminus of the first subunit of the Fc knob domain, and wherein the second antigen-binding moiety is fused at the C-terminus of the Fab heavy chain (CH1) to the N-terminus of the second subunit of the Fc hole domain.

The bispecific antibody of the present invention uses CROSSFAB approach, which crossovers the constant domain and variable domain and switches the CH1 domain and CL domain in the CD3e Fab molecule, which reduces undesired mis-paring.

In one embodiment, the bispecific antibody of the present invention comprises: (1) humanized PLAP light chain, (2) CD3e cross FAB, CD3VL-CH1; (3) humanized PLAP VH-CH1-CD3e CROSSFAB (VH-CL)—Fc (knob), and (4) humanized PLAP VH-CH1—Fc (hole). (FIG. 1A)

In one embodiment, the VH of the humanized PLAP antibody has the amino acid sequence of SEQ ID NO: 10 and the VL has the amino acid sequence of SEQ ID NO: 4.

In another embodiment, the VH of the humanized PLAP antibody has the amino acid sequence of SEQ ID NO: 19 and the VL has the amino acid sequence of SEQ ID NO: 16.

In one embodiment, the Fc domain comprises a modification promoting the association of the first and the second subunit of the Fc domain.

In one embodiment, in the CH3 domain of the first subunit of the Fc domain, an amino acid residue is replaced with an amino acid residue having a larger side chain volume, thereby generating a protuberance within the CH3 domain of the first subunit which fits in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit fits.

In one embodiment, the Fc domain exhibits reduced binding affinity to an Fc receptor and/or reduced effector function, as compared to a native IgG Fc domain.

In one embodiment, the Fc domain comprises one or more amino acid substitution that reduces binding to an Fc receptor and/or effector function. In one embodiment, the one or more amino acid substitution in the Fc domain are selected from the group of L234, L235, and P329 (Kabat numbering). In one embodiment, said amino acid substitutions are L234A, L235A and P329G.

In one embodiment, silent Fc mutations P329G, and L234A and L235A mutations are used to prevent Fc-dependent immune reactions.

In one embodiment, only silent mutations L234A and L235A mutations are used to prevent Fc-dependent immune reactions.

In a specific embodiment, the Fc domain is modified with a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. No. 5,731,168. Generally, the method involves introducing a protuberance (“knob”) at the interface of a first polypeptide and a corresponding cavity (“hole”) in the interface of a second polypeptide, such that the protuberance can be positioned in the cavity to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

In one embodiment, a “knob” is made by mutations of S354C and T366W on one Fc, and the corresponding “hole” is made by mutations of Y349C, T366S, L368A and Y407V on the partner Fc.

In one embodiment, the bispecific antigen-binding molecule comprising two binding moieties to PLAP, and one binding moiety to CD3 epsilon, the molecules comprises the amino acid sequences of SEQ ID NO: 5, 8, 12, and 14, in a molar ratio of 2:1:1:1; optionally each amino acid sequence has at least 95%, 96%, 97%, 98%, or 99% sequence identity thereof, provided that the sequence variation is in the non-CDR framework regions.

In one embodiment, the bispecific antigen-binding molecule comprising two binding moieties to PLAP, and one binding moiety to CD3 epsilon, the molecules comprises the amino acid sequences of SEQ ID NO: 17, 8, 20, and 22, in a molar ratio of 2:1:1:1; optionally each amino acid sequence has at least 95%, 96%, 97%, 98%, or 99% sequence identity thereof, provided that the sequence variation is in the non-CDR framework regions.

### Bispecific Antibody Structure of FIG. 1B

FIG. 1B shows the structure of humanized bivalent bispecific PLAP-CD3e antibody consisting of 3 DNA constructs. This structure comprises two binding moieties to PLAP and one binding moiety to CD3 epsilon.

In one embodiment, the antibody comprises the amino acid sequences of SEQ ID NO: 17, 24, and 22, in a molar ratio of 2:1:1; optionally each amino acid sequence has at least 95%, 96%, 97%, 98%, or 99% sequence identity thereof, provided that the sequence variation is in the non-CDR framework regions.

### Bispecific Antibody Structure of FIG. 1C

FIG. 1C shows a bispecific antibody structure of monovalent humanized PLAP and monovalent CD3e; the structure consists of 3 DNA constructs. The structure does not have CD3 CROSS FAB, but is has a CD3e scFv. The bispecific antibody comprises one binding moiety to PLAP, and one binding moiety to CD3 epsilon.

In one embodiment, the bispecific antibody comprises the amino acid sequences of SEQ ID NO: 5, 28, and 30, in a molar ratio of 2:1:1; optionally each amino acid sequence has at least 95%, 96%, 97%, 98%, or 99% sequence identity thereof, provided that the sequence variation is in the non-CDR framework regions.

In another embodiment, the bispecific antibody comprises the amino acid sequences of SEQ ID NO: 17, 28, and 30, in a molar ratio of 2:1:1; optionally each amino acid sequence has at least 95%, 96%, 97%, 98%, or 99% sequence identity thereof, provided that the sequence variation is in the non-CDR framework regions.

The above sequence variations of structures of FIG. 1A-1C, i.e., the amino acid changes are preferably of a minor amino acid change such as a conservative amino acid substitution. A conservative amino acid substitution is well-known to a person skilled in the art.

The present invention is directed to a bispecific antibody method for treating cancer, comprising the step of administering PLAP-CD3e antibody to a subject suffering from cancer, wherein the cancer is selected from the group consisting of colon cancer, lung cancer, pancreatic cancer, stomach cancer, testicular cancer, teratoma, seminoma, ovarian cancer, and cervical cancer, and the cancer is PLAP-positive.

The present invention is also directed to a pharmaceutical composition comprising the bispecific antigen-binding molecule and a pharmaceutically acceptable carrier.

The nucleic acid encoding the bispecific antibody of the present invention can be inserted into a vector and expressed in mammalian 293S or CHO cells using serum-free medium. The antibody can be purified with protein A or protein G column and used for the study.

This application demonstrates the efficacy of bispecific antibody targeting PLAP antigen that is overexpressed in colon cancer tumors. This application demonstrates that PLAP-CD3e antibody binds CD3e antigen and PLAP antigen. This antibody delivered with T cells specifically decreases viability of PLAP-positive colon cancer cells but not PLAP-negative cancer cells. PLAP-CD3e antibody delivered with T cells caused secretion of significant level of IFN-gamma after co-incubation with PLAP-positive colon cancer cells but not after co-incubation with PLAP-negative cancer cells. This application demonstrates that PLAP-CD3e antibody administered with T cells significantly decreased Lovo (positive PLAP-colon cancer cells) xenograft tumor growth in vivo.

The inventors demonstrate that PLAP-CD3 antibody with T cells significantly killed all PLAP-positive cancer cells, but not kill PLAP-negative colon cancers. This implies high specificity of PLAP-CD3 antibody.

The inventors demonstrated high efficacy of three different designs of bispecific antibodies of FIGS. 1A-1C.

The following examples further illustrate the present invention. These examples are intended merely to be illustrative of the present invention and are not to be construed as being limiting.

### EXAMPLES

### Example 1

### Materials and Methods

### Cells and Culture Medium

HEK293FT cells from A/Stem (Richmond, Calif.) were cultured in Dulbecco's Modified Eagle's Medium (DMEM) plus 10% FBS and 1% penicillin/streptomycin. Human peripheral blood mononuclear cells (PBMC) were isolated from whole blood obtained from the Stanford Hospital Blood Center, Stanford, Calif. according to IRB-approved protocol using Ficoll-Paque solution (GE Healthcare). Colon cancer cell lines: PLAP-negative: HT29, and PLAP-positive: Lovo cells were used for the study. The cells were cultured in a humidified 5% CO₂ (9).

### Antibodies

The (APC)-labeled anti-CD3 and secondary antibodies were described in (9).

### PLAP-CD3 Antibody Constructs

The four constructs of Example 2A were designed according to Cross-Fab designed described in (10). The constructs had P329G mutation and Leucine 324,235 changed to alanine, called LA-LA to decrease Fc immune activity. In addition, Fc silent and knobs-in-hole mutations were used for engineering, as described (10). We also expressed three constructs of FIG. 1B and three constructs of FIG. 1C. All constructs for FIG. 1A and FIG. 1B were cloned into Nhe I and Nsi I sites of pYD11 vector.

### Expression of PLAP-CD3 Antibodies

For structure FIG. 1A, the four antibody constructs were mixed at weight ratio 2 (PLAP VL-CL):1:1:1 (μg/mL) with NanoFect transfection agent and used for 293S cell transformation. For structures 1B and 1C, the three antibody constructs were mixed at weight ratio 1:1:1 (μg/mL) with NanoFect transfection agent and used for 293S cell transformation. The cells were rotated in bottles on shaker in Freestyle F17 medium, containing 8 mM L-Glutamine (or GlutaMAX), and 0.1% Pluoronic F-68 for one week at 37° C. incubator. The supernatant or purified antibody on protein A column was analyzed on SDS gel, by FACS and functional assays.

### PBMC

PBMC were resuspended at 1×10⁶ cells/ml in AIM V-AlbuMAX medium (Thermo Fisher) containing 10% FBS with 300 U/ml IL-2 (Thermo Fisher). PBMC cells were activated with CD3/CD28 Dynabeads (Invitrogen), and used for cytotoxicity analysis with bi-specific antibodies.

### Fluorescence-Activated Cell Sorting (FACS) Analysis

The allophycocyanin (APC)-labeled anti-CD3 (eBioscience, San Diego, Calif.) antibody was used for FACS analysis using FACSCalibur (BD Biosciences). For FACS with colon cancer cell lines to detect PLAP levels, either bi-specific PLAP-CD3 or mouse monoclonal PLAP antibody (H17E2) from Ximbio (London, UK) were used for FACS analysis which was performed on FACSCalibur, as described (9).

### Real-Time Cytotoxicity Assay (RTCA)

Adherent colon cancer target cells (10,000 cells per well) were seeded into 96-well E-plates (Acea Biosciences, San Diego, Calif.) and cultured overnight using the impedance-based real-time cell analysis (RTCA) iCELLigence system (Acea Biosciences). After 20-24 hours, the medium was replaced with 1×10⁵ effector cells T cells, T cells with bispecific antibody or antibody alone in AIM V-AlbuMAX medium containing 10% FBS, in triplicate. The cells were monitored for >40 hours with the RTCA system, and impedance (proportional to cell index) was plotted over time. Cytotoxicity was calculated as (impedance of target cells without effector cells—impedance of target cells with effector cells)×100/impedance of target cells without effector cells.

### ELISA Assay for Cytokine Secretion

The target cells were cultured with the effector cells or agents at in U-bottom 96-well plates with AIM V-AlbuMAX medium plus 10% FBS, in triplicate. After 16 h the supernatant was removed and centrifuged to remove residual cells. In some experiments, supernatant after RTCA assay was used for ELISA cytokine assays. The supernatant was transferred to a new 96-well plate and analyzed by ELISA for human cytokines using kits from Thermo Fisher according to the manufacturer's protocol.

### Mouse in vivo Xenograft Study

Six-week old male NSG mice (Jackson Laboratories, Bar Harbor, Me.) were housed in accordance with the Institutional Animal Care and Use Committee (IACUC) protocol. Each mouse was injected subcutaneously with 2×10⁶ Lovo colon cancer cells in sterile lx PBS. The bi-specific antibody 10 μg/mice with 1×10⁷ T cells were injected intravenously into mice at different time points. Tumor sizes were measured with calipers twice weekly and tumor volume (in mm³) was determined using the formula W²L/2, where W is tumor width and L is tumor length. At the end 0.1 ml of blood was collected and used for analysis of toxicology markers.

### Example 2

### The Sequence of PLAP H2-CD3E Bispecific Antibody (FIG. 1A)

FIG. 1A shows the structure of humanized PLAP-CD3 bivalent antibody consisting of 4 DNA constructs. The structure has CD3 CROSS-Fab.

PLAP h2-CD3e bispecific antibody of FIG. 1A comprises 4 constructs:
1. PLAP h2 light chain (VL-CL): PLAP VL (humanized h2 PLAP, WO2019/240934, which was codon optimized as below)
2. CD3 CROSSFAB, (VL-CH1)
3. PLAP h2 VH-CH1-CD3 CROSSFAB (VH-CL)—Fc (knob) P329GLA-LA
4. PLAP h2 VH-CH1—Fc(hole) P329GLA-LA

P329G mutation abolishes interaction of FcγR and C1q interactions and thus eliminates elimination of targeted cells via antibody-dependent cellular-cytotoxicity (ADCC), antibody-dependent phagocytosis (ADCP) or complement-dependent cytotoxicity (CDC). P329G mutation removes FcγR-mediated immune effector functions when delivered to cells providing silent Fc region (11). Addition of two other mutations LA-LA mutation changes Leucine Leu 234 and Leu 235 to alanine (A) completely blocked binding of FcγR and C1q interactions and thus abolished Fc-mediated ADC, ADCC and other immunogenicity (10).

All sequences were codon optimized and synthesized as GBlocks and inserted into Nhe I and Nsi I site of pYD11 vector. In order not to have mispairing of light chain domains, CrossFAB technology was used whereCD3 VH is connected to CL, and CD3 VL is connected to CH1. We also used knobs-in-hole mutations proposed by Crick in 1952 in order to create the knob (T366W), and S354C mutations were used; or the hole (Y349C, T366S, L368A and Y407V) mutations were used to hold both Fc chains together. All sequences start with the signaling peptide (underlined): METDTLLLWVLLLWVPGSTGAAS (SEQ ID NO: 2).

### Construct #1. PLAP h2 Light Chain: LC-PLAP

DNA artificial sequence LC (light chain) of humanized PLAP (PLAP h2 VL (bold)-CL (italics) is shown below. The nucleotide sequence of PLAP h2 VL is shown in WO2019/240934 which was codon-optimized and inserted with constant CL region into Nhe I (GCTAGC site shown in italics, underlined) and Nsi I sites (atgcat shown in italics, underlined of pYD11 vector). The sequences started with signaling peptide (Signaling peptide is underlined+(AAS amino-acids after due to cloning site): METDTLLLWVLLLWVPGSTGAAS (SEQ ID NO: 2).

Two stop codons were added to the sequence before start of human Fc to express light chain with no Fc present in the vector. Signaling peptide in bold italics, underlined; VL bold; CL italics.

(SEQ ID NO: 3)

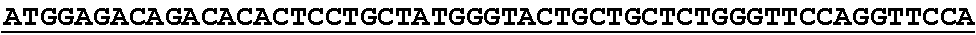

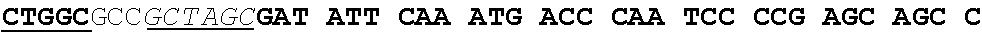

TG AGT GCC AGC GTA GGA GAT CGG GTT ACG ATA ACG TGTCGA G
CA TCC GAA AAT ATC TAT AGC TAT GTA GCA TGG TAC CAA CAA
AAA CCT GGC AAG GCA CCG AAA CTGTTG ATA TAC AAC GCC AAG
AGT CTG GCA AGT GGG GTG CCT TCA CGC TTC AGT GGA AGC GGA
AGT GGG ACAGAT TTT ACA CTT ACC ATT TCC TCC CTT CAG CCT
GAA GAC TTT GCA ACA TAT TAT TGT CAA CAT CAC TATGTG AGT
CCG TGG ACT TTC GGG GGA GGA ACC AAA TTG GAA ATA AAG CG
C ACA GTC GCA GCG CCC AGT GTGTTT ATA TTC CCC CCT TCA GA
C GAA CAG CTC AAA TCC GGC ACA GCA TCA GTG GTG TGT CTG T
TG AAC AATTTC TAT CCT AG A GAG GCA AAG GTT CAA TGG AAA G
TG GAC AAC GCG CTC CAA AGC GGG AAC TCC CAA GAGAGC GTC A
CT GAA CAA GAT TCC AAA GAT AGC ACG TAC TCT CTT TCT TCA
ACG CTC ACG CTC AGT AAG GCCGAT TAC GAG AAA CAT AAG GTA
TAC GCT TGC GAG GTC ACG CAT CAA GGG CTG TCC TCA CCC GTG

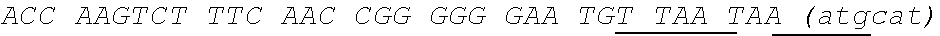

Amino-acid sequence:
Signaling peptide (underlined) + AAS:
(SEQ ID NO: 2)
METDTLLLWVLLLWVPGSTGAAS
PALP h2 VL
(SEQ ID NO: 4)
DIQMTQSPSSLSASVGDRVTITCRASENIYSYVAWYQQKPGKAPKLLIYNAKSLASG
VPSRFSGSGSGTDFTLTISSLQPEDFATYYCQHHYVSPWTFGGGTKLEIKR
PALP h2 VL-CL
(SEQ ID NO 5)
DIQMTQSPSSLSASVGDRVTITCRASENIYSYVAWYQQKPGKAPKLLIYNAKSLASG
VPSRFSGSGSGTDFTLTISSLQPEDFATYYCQHHYVSPWTFGGGTKLEIKRTVAAPSV
FIFPPSDEQLKSGTASWCLLNNFYPREAKVQWKVDNALQSGNSQESVTEQDSKDSTYSLSSTL
TLSKADYEKHKVYACEVTHQGLSSPVTKSFNRGEC

### Construct #2. CD3 CROSSFAB (VL-CH1)

CD3 VL is shown in bold, CH1 is in italics font, the nucleotide sequence was codon optimized. The Nhe I and Nsi I sites are shown in italics. The stop codon TAA was added to terminate the sequence before Fc.

Nucleotide sequence: Signaling peptide underlined in italics in bold, then AAS in italics regular font; VL in bold, CH1, regular font italics.

(SEQ ID NO: 6)

[symbol]

[symbol]

[symbol]

[symbol]

[symbol]

GCCGCTAGCCAG GCC GTA GTG ACA CAG GAA CCG TCT TTG A
CG GTG TCT CCG GGA GGT ACC GTC ACC TTG ACG TGT GGGTCC A
GC ACT GGA GCT GTA ACA ACG AGC AAT TAC GCG AAT TGG GTG
CAG GAG AAG CCA GGT CAG GCT TTTAGG GGT CTT ATC GGA GGG
ACT AAT AAA AGG GCT CCA GGC ACG CCG GCA AGA TTC TCA GGG
TCC CTG CTGGGG GGG AAA GCG GCA CTC ACC CTT TCT GGT GCT
CAG CCA GAG GAT GAG GCC GAA TAT TAT TGT GCC TTGTGG TAT
TCT AAT TTG TGG GTC TTT GGA GGC GGG ACA AAA CTC ACT GT
A TTG TCA TCT GCG TCA ACG AAGGGA CCT TCT GTA TTC CCC TT
G GCA CCA TCC AGT AAA TCT ACC AGT GGG GGT ACC GCT GCC C
TC GGT TGCCTT GTA AAA GAT TAC TTT CCG GAG CCC GTC ACC G
TG TCC TGG AAC AGC GGG GCA TTG ACC AGT GGT GTCCAC ACT T
TT CCC GCA GTA CTC CAA AGC TCC GGC CTC TAC AGT CTC TCT
TCA GTT GTG ACG GTT CCT AGCTCT TCC CTT GGT ACG CAG ACT
TAT ATC TGC AAC GTC AAC CAC AAA CCT TCC AAT ACT AAG GTA
GAC AAAAAG GTG GAG CCC AAA TCT TGT [symbol] ATGCAT
Amino-acid sequence (not including signaling peptide)
CD3 VL
(SEQ ID NO: 7)
QAVVTQEPSLTVSPGGTVTLTCGSSTGAVTTSNYANWVQEK
PGQAFRGLIGGTNKRAPGTPARFSGSLLGGKAALTLSGAQP
EDEAEYYCALWYSNLWVFGGGTKLTVL
CD3 VL-CH1
(SEQ ID NO: 8)
QAVVTQEPSLTVSPGGTVTLTCGSSTGAVTTSNYANWVQEK
PGQAFRGLIGGTNKRAPGTPARFSGSLLGGKAALTLSGAQP
EDEAEYYCALWYSNLWVFGGGTKLTVLSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC

### Construct #3

### PLAP h2 VH CH1-CD3 CROSSFAB VH-CL—Fc (Knob) P329GLA-LA

Signaling peptide in bold, italics underlined, then 3 amino acids-AAS due to cloning sites; Cloning sites Nhe I GCTAGC and Nsi I ATGCAT are underlined, larger font

PLAP h2-VH-in bold; CH1-underlined; 2×G4S linker; CD3 VH bold italics; CL in italics underlined; IgG Fc chain with LA-LA, (L234 and L235 changed to A) mutations shown in bold, underlined, and P329G mutation, P changed to G, bold underlined. The knob mutations in Fc domain were S354C and T366W shown in bold larger font, italics.

Nucleotide sequence:
(SEQ ID NO: 9) [symbol] [symbol] [symbol] [symbol]
GCCGCTAGCCAG GT
A CAA TTG CAG GAA TCA GGA CCC GGA TTG GTG AAG CCA AGT GAA ACT CT
G AGT TTG ACT TGC ACAGTC AGC GGC TTC TCA CTG ACA TCC TAT GGG GTA
AGT TGG ATT CGG CAA CCG GCA GGT AAG GGC CTT GAATGG ATC GGG GTC
ATC TGG GAA GAT GGG TCA ACT AAC TAC CAT TCT GCG CTG ATA AGT CGC
GTT ACC ATGTCA GTC GAT ACA AGC AAG AAT CAG TTC TCT CTG AAA CTG A
GC AGT GTG ACA GCC GCA GAC ACC GCA GTGTAC TAT TGC GCA CGC CCT CA
C TAC GGA TCC TCT TAT GTG GGA GCA ATG GAA TAT TGG GGA GCG GGA AC
AACA GTA ACA GTT TCT TCA GCG AGC ACT AAG GGC CCG TCT GTA TTT CCC
CTT GCC CCT TCA TCC AAG AGCACG AGT GGT GGA ACG GCC GCA CTC GGA
TGT TTG GTA AAA GAC TAT TTC CCA GAG CCC GTG ACT GTG TCTTGG AAT T
CC GGT GCA CTG ACT TCT GGT GTG CAT ACT TTT CCG GCC GTG CTT CAA A
GT TCA GGC CTT TATAGC TTG AGC TCA GTA GTC ACC GTC CCT TCA TCT TC
A TTG GGG ACA CAA ACC TAT ATC TGT AAT GTT AATCAT AAA CCT TCC AAC
ACC AAA GTC GAC AAG AAA GTG GAG CCA AAG ACT TGC GAT GGG GGA GGG
GGA AGCGGG GGA GGG GGT TCA GAG GTA TCA GAG GTA CAG CTG CTC GAA AGC GGC GGA
GGT CTT GTG CAA CCA GGC GGG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GCA TCA GTT GCG GCC CCC TCA GTCTTC ATT TTT CCC CCT AG
T GAT GAG CAA CTT AAG TCC GGA ACA GCC AGC GTG GTC TGC CTG CTG AA
C AATTTT TAT CCG AGG GAG GCG AAG GTT CAA TGG AAA GTC GAT AAC GCT
CTG CAA TCA GGT AAT TCT CAG GAATCT GTC ACT GAA CAA GAT AGT AAA
GAC AGC ACA TAC TCT TTG TCT TCT ACA TTG ACC TTG TCT AAG GCGGAT T
AC GAA AAG CAT AAG GTC TAT GCT TGC GAA GTG ACG CAT CAG GGG CTT A
GT TCC CCG GTC ACC AAGAGT TTC AAT AGG GGG GAG TGC GAT AAG ACC CA
C ACC TGT CCG CCA TGC CCT GCA CCT GAG GCA GCG GGAGGG CCG AGT GTA
TTC TTG TTC CCT CCA AAA CCG AAA GAT ACT CTG ATG ATT AGC CGG ACC
CCC GAA GTTACG TGT GTG GTT GTA GAC GTA AGT CAC GAA GAT CCT GAA
GTT AAG TTT AAC TGG TAT GTT GAT GGG GTGGAA GTT CAC AAT GCC AAA A
CC AAA CCT AGA GAG GAG CAA TAC AAC TCC ACC TAT CGG GTT GTA AGC G
TCTTG ACC GTG CTC CAC CAA GAC TGG CTG AAC GGT AAG GAG TAT AAG TG
T AAG GTG AGC AAC AAG GCT TTGGGA GCA CCC ATC GAA AAA ACG ATC AGC
AAA GCC AAA GGT CAG CCA GCG GAA CCC CAG GTG TAT ACC CTTCCG CCT [symbol]
AGG GAT GAG CTT ACT AAG AAC CAA GTT TCA CTC [symbol] TGT CTG GTG
AAG GGT TTT TAC CCCTCC GAT ATT GCT GTG GAG TGG GAG TCA AAC GGG
CAG CCA GAA AAT AAC TAT AAG ACC ACG CCA CCT GTCCTT GAC AGT GAC G
GA AGT TTT TTC CTG TAT TCT AAA TTG ACC GTA GAT AAG TCT CGA TGG C
AG CAA GGAAAC GTG TTT TCA TGC TCT GTT ATG CAC GAA GCT CTC CAC AA
C CAT TAT ACA CAA AAG TCA CTG AGC CTTAGT CCT GGT AAAATGCATGAGGCT
CTGCACAACCACTACACGCAGAAGAGCCTCTCCCTGTCTCCCGGGAAATGA
(SEQ ID NO: 9).
Amino-acid sequence PLAP h2 VH,
SEQ ID NO: 10
QVQLQESGPGLVKPSETLSLTCTVSGFSLTSYGVSWIRQPAGKGLEWIGVIWEDGS
TNYHSALISRVTMSVDTSKNQFSLKLSSVTAADTAVYYCARPHYGSSYVGAMEYW
GAGTTVTVSS
CD3 VH,
SEQ ID NO: 11

[symbol]

[symbol]

[symbol]

[symbol]

[symbol]

[symbol]

[symbol]

[symbol]

[symbol]

Amino-acid sequence of Construct #3 (not including signaling peptide):
(SEQ ID NO: 12)
QVQLQSGPGLVKPETLSLTCTVSGFSLTSYGVSWIRQPAGKGLEWIGV
IWEDGSTNYHSALISRVTMSVDTSKNQFSLKLSSVTAADTAVYYCARPHY
GSSYVGAMEYWGAGTTVTVSSASTKGPSVFPLAPSSKSTSGGTAALGCLVK
DYFPEPVTVSWNSGALTSGVHTFPAVLQSSGLYSSVVTVPSSSLGTQTYICN
VNHKPSNTKVDKKVEPKSCDGGGGSGGGGS

[symbol]

[symbol]

[symbol]

[symbol]

[symbol]

[symbol]

[symbol]

[symbol]

ASVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGECDKTH
TCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LGAPIEKTISKAKGQPREPQVYTLPP [symbol] RDELTKNQVSL [symbol] CLVKGFYPSDIAVE
WESNGQPENNYKTTPPVLDSDGSFFLYSKLTVDKSRWQQGNVFSCSVMHEA
LHNHYTQKSLSLSPGKMHEALHNHYTQKSLSLSPGK

### Construct #4. PLAP h2 VH-CH1—Fc(hole) P329GLA-LA

Construct #4 used the same P329G and LA-LA mutations as in Construct #3, shown in bold. The hole mutations were Y349C, T366S, L368A and Y407V shown in bold, larger fond, italics. Cloning sites Nhe I GCTAGC and Nsi I ATGCAT are underlined

Signaling peptide underlined, bold, italics, then 9 nucleotides encoding 3 amino-acids AAS (cloning sites), regular font, italics; PLAP-VH-bold, CH1 underlined, then Fc with P329GLA-LA and hole mutations

Nucleotide:
(SEQ ID NO: 13)

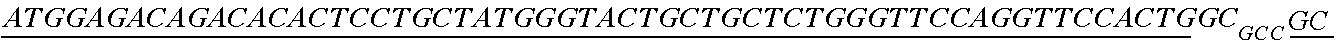

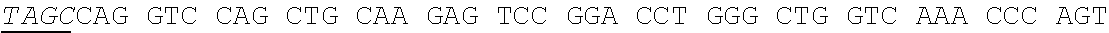

GAG ACG TTG TCC CTT ACG TGT ACTGTC TCC GGC TTC AGT TTG ACG TCT T
AT GGA GTT TCT TGG ATA CGG CAG CCC GCC GGT AAG GGC CTC GAGTGG AT
T GGA GTT ATA TGG GAA GAT GGG TCC ACT AAT TAT CAT AGC GCC CTT AT
T AGC AGG GTA ACC ATGTCT GTC GAT ACT AGC AAA AAT CAG TTC AGC CTT
AAA TTG TCA AGT GTG ACC GCT GCA GAT ACA GCA GTATAT TAC TGT GCG
AGA CCA CAT TAT GGA TCC AGT TAT GTC GGA GCG ATG GAG TAT TGG GGC

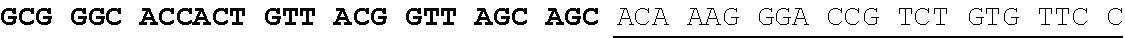

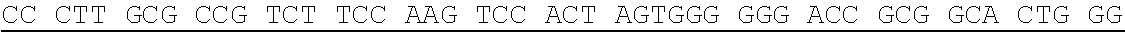

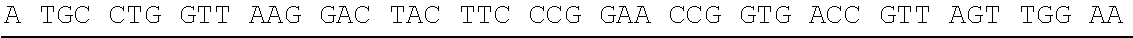

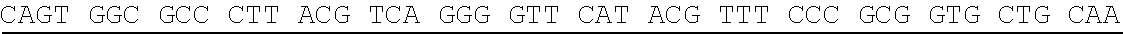

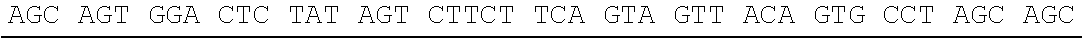

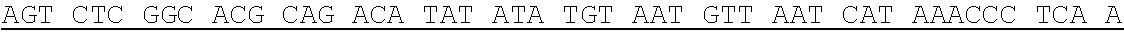

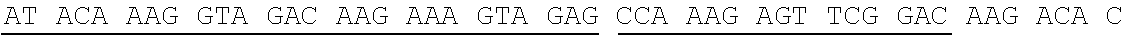

AT ACT TGC CCT CCGTGC CCT GCA CCC GAA GCG GCA GGC GGC CCA TCA GT
A TTT TTG TTT CCT CCT AAA CCT _AAA GAC ACT CTTATG ATA TCA CGG ACA
CCT GAA GTC ACT TGT GTA GTT GTG GAC GTT TCA CAT GAG GAT CCC GAA
GTC AAGTTC AAC TGG TAC GTC GAT GGC GTA GAA GTT CAT AAC GCA AAA
ACA AAG CCG CGG GAG GAG CAG TAT AACTCA ACC TAT CGA GTA GTC TCT G
TT CTT ACG GTT TTG CAT CAA GAC TGG CTC AAT GGT AAG GAG TAT AAATG

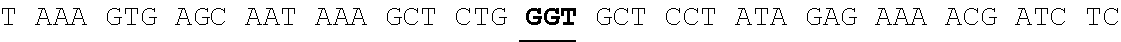

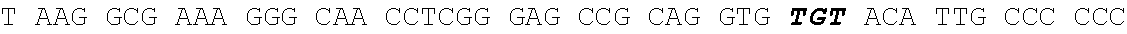

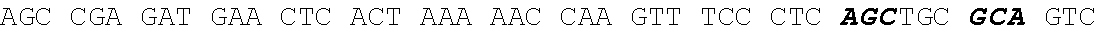

AAG GGA TTC TAC CCG AGC GAC ATT GCA GTA GAG TGG GAG TCA AAT GGT
CAG CCA GAA AATAAT TAT AAA ACA ACC CCT CCC GTC CTG GAC AGC GAT

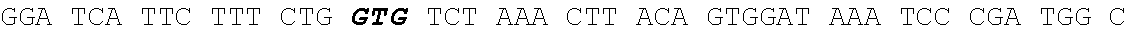

AA CAA GGC AAT GTG TTT TCT TGT TCT GTA ATG CAC GAG GCA TTG CAT A

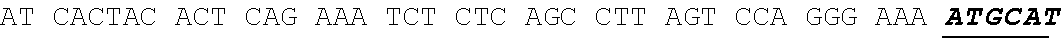

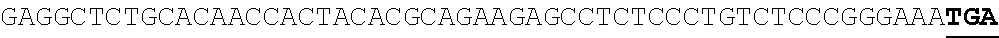

Amino-acid of Construct #4 (not including signaling peptide),
SEQ ID NO: 14
QVQLQESGPGLVKPSETLSLTCTVSGFSLTSYGVSYVIRQPAGKGLEWIGVIWEDGS
TNYHSALISRVTMSVDTSKNQFSLKLSSVTAADTAVYYCARPHYGSSYVGAMEYW

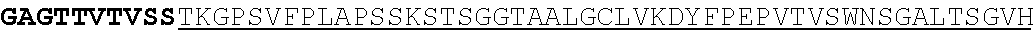

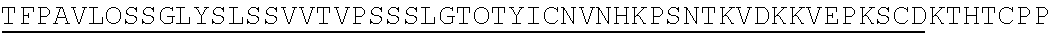

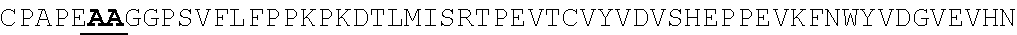

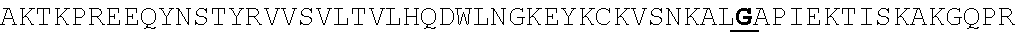

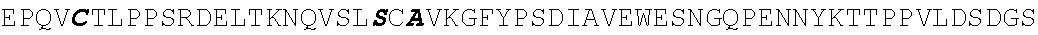

FFLVSKLTVDKSRWQQGNVFSCSVMHEALHNHYTQKSLSLSPGKMHEALHNHYTQKS
LSLSPGK

### Example 3

### the Sequence of PLAP H4-CD3 Antiboty (FIG. 1A)

PLAP h4-CD3e bispecific comprises 4 constructs:
1. PLAP h4 light chain (VL-CL): LC PLAP
2. CD3 CROSSFAB, (CD3e VL-CH1), same as Example 2.
3. PLAP h4 VH-CH1-CD3 CROSSFAB (CD3e VH-CL)—Fc (knob) P329GLA-LA
4. PLAP h4 VH-CH1-Fc(hole) P329G, LA-LA

### Construct #1

PLAP h4 light chain: LC PLAP (humanized h4 PLAP, WO2019/240934, which was codon optimized as below), signaling peptide in bold, italics, underlined; followed by 9 nucleotides, cloning sites in italics regular font; Nhe I and Nsi I sites underlined. PLAP h4 VL is shown in bold, then CL in regular font

Nucleotide sequence:
(SEQ ID NO: 15) [symbol] [symbol] [symbol]
GCCGCTAGCGAC
ATACAG ATG ACT CAA AGC CCC TCT TCA CTG TCT GCA TCA GTC GGG GAC
AGA GTC ACA ATA ACC TGC AGA GCGAGC GAG AAT ATC TAC TCT TAT GTA
GCC TGG TAT CAG CAA AAA CCC GGC AAG GCG CCG AAA TTG CTC ATCTAT
AAT GCG AAA TCC TTG GCC AGT GGG GTC CCA TCA CGG TTC AGT GGC TCC
GGC TCT GGA ACC GAT TTCACA CTC ACA ATC TCT AGC CTC CAG CCC GAA
GAC TTC GCC ACA TAC TAT TGC CAA CAT CAC TAT GTC AGCCCA TGG ACA
TTT GGG GGA GGT ACG AAA CTT GAA ATT AAACGT ACA GTA GCT GCT CCG
TCC GTC TTT ATT TTC CCG CCG TCT GAC GAA CAG CTC AAA AGC GGG ACT
GCATCA GTT GTC TGT CTC CTC AAC AAT TTT TAC CCG CGA GAG 20A ACTT GTT
CAA TGG AAA GTT GAT AAC GCCCTC CAG AGT GGA AAC TCT CAG GAG AGT GTA
ACT GAG CAA GAT TCC AAA GAT TCA ACC TAT AGT CTT TCAAGT ACC TTG ACT
CTT TCT AAA GCG GAT TAT GAG AAA CAT AAA GTG TAT GCC TGC GAA GTG ACC
CAT CAGGGG CTT TCA TCA CCC GTG ACG AAG TCC 20A ACTT CGA GGC GAA
TGC TAAATGCAT
Amino-acid sequence of PLAP h4 VL,
SEQ ID NO: 16
DIQMTQSPSSLSASVGDRVTITCRASENIYSYVAWYQQKPGKAPKWYNAKSLASG
VPSRFSGSGSGTDFTLTISSLQPEDFATYYCQHHYVSPWTFGGGTKLEIKR
Amino-acid sequence of Construct #1, PLAP h4 VL-CL (without signaling peptide),
SEQ ID NO: 17
DIQMTQSPSSLSASVGDRVTITCRASENIYSYVAWYQQKPGKAPKWYNAKSLASG
VPSRFSGSGSGTDFTLTISSLQPEDFATYYCQHHYVSPWTFGGGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC

### Construct #2 (Light Chain of CD3 CrossFAB) Was Same as Example 2

### Construct #3 PLAP h4 VH-CH1-CD3 CROSSFAB (VH-CL)—Fc (Knob)

Signaling peptide in italics, underlined, in bold, with following 9 nucleotides (cloning sites); PLAPh4 VH in bold; CH1 underlined; CD3 VH bold in italics; CL in italics, underlined; Fc with P329GLA-LA mutations in bold underlined: knob mutations in bold italics, larger font.

(SEQ ID NO: 18) [symbol] [symbol] [symbol]
GCCGCTAGCCAG GT
T CAA CTT CAA GAA TCA GGA CCG GGC TTG GTT AAA CCT TCC GAA ACT CT
G AGC CTT ACT TGT ACAGTG TCT GGT GGA TCT ATT ACG AGC TAC GGA GTA
AGT TGG ATC CGG CAA CCA CCC GGG AAA GGG CTC GAATGG ATA GGG GTG
ATA TGG GAG GAT GGT TCA ACC AAC TAC CAT AGC GCT CTG ATC AGC CGG
GTG ACC ATTAGT GTC GAC ACT TCC AAA AAC CAG TTT TCA TTG AAG CTC T
CA AGC GTA ACT GCG GCG GAT ACC GCC GTATAC TAT TGT GCG CGG CCA CA
T TAC GGG TCC TCT TAT GTT GGG GCG ATG GAA TAT TGG GGG GCA GGT AC
AACG GTC ACG GTG TCT TCA ACA AAA GGT CCT TCC GTA TTT CCG CTC GCA
CCC AGC TCT AAG TCA ACC TCT GGC GGT ACT GCA GCC CTG GGT TGC CTC
GTA AAG GAC TAT TTT CCT GAG CCA GTA ACA GTT TCT TGG AAC AGC GGG
GCA CTT ACG AGC GGT GTT CAT ACG TTC CCT GCA GTG TTG CAA TCC AGC
GGC CTT TAT TCA TTG TCT TCA GTT GTA ACG GTT CCT TCT AGT AGT TTG
GGG ACC CAG ACA TAT ATC TGC AAC GTG AAC CAT AAG CCA AGC AT ACC
AAA GTT GAT AAG AAG GTC GAA CCT AAG TCC TGC GAC GGC GGG GGA GGA
TCT GGC GGG GGA GGC AGT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GCC AGT GTA GCG GCC CCG TCC GTT TTC ATA TTC CCT CCT
TCC GAC GAG CAG TTG AAA AGC GGT ACG GCG AGC GTT GTG TGC TTG TTG
AAC AAC TTC TAC CCA CGC GAA GCC AAG GTC CAA TGG AAG GTA GAC AAC
GCA CTG CAG AGT GGT AAC TCA CAG GAA TCA GTG ACG GAA CAG GAC TCA
AAA GAT AGT ACT TAC AGT CTT TCT TCC ACA CTG ACA CTC AGT AAG GCC
GAT TAT GAG AAA CAT AAA GTA TAC GCA TGT GAA GTA ACT CAC CAG GGT
CTC AGT TCA CCA GTA ACT AAG TCT TTC AAT CGC GGG GAA TGC GAC AAA
ACA CAC ACC TGT CCC CCC TGT CCA GCC CCA GAG GCA GCT GGC GGC CCT
AGT GTG TTC TTG TTC CCG CCC AAG CCA AAA GAT ACA CTG ATG ATT AGC
CGG ACC CCT GAG GTA ACT TGT GTG GTG GTG GAC GTG TCT CAT GAG GAC
CCA GAG GTA AAA TTC AAC TGG TAC GTA GAC GGC GTC GAG GTC CAT AAT
GCC AAA ACC AAG CCA CGG GAG GAG CAG TAT AAT TCC ACT TAT CGC GTA
GTC TCT GTA CTT ACA GTT CTT CAC CAA GAT TGG TTG AAC GGA AAA GAA
TAC AAG TGT AAA GTT AGC AAT AAG GCG CTC GGA GCT CCG ATC GAA AAA
ACA ATC TCC AAA GCA AAA GGG CAA CCC CGA GAA CCA CAG GTA TAC ACC
CTG CCG CCG [symbol] CGA GAC GAG CTG ACG AAA AAC CAA GTG TCC CTG [symbol]
TGC TTG GTG AAG GGC TTT TAT CCA AGT GAC ATT GCA GTT GAA TGG GAG
TCT AAC GGA CAG CCT GAA AAT AAC TAT AAG ACC ACG CCA CCA GTC CTT
GAT AGC GAT GGA TCT TTT TTT CTC TAT AGC AAG TTG ACT GTA GAT AAA
TCA CGA TGG CAA CAA GGC AAT GTC TTT TCA TGC AGC GTT ATG CAT
GAGGCTCTGCACAACCACTACACGCAGAAGAGCCTCTCCCTGTCTCCCGGGAAATGA
Amino Acid Sequence of PLAP h4 VH,
SEQ ID NO: 19
QVQLQESGPGLVKPSETLSLTCTVSGGSITSYGVSWIRQPPGKGLEWIGVIWEDGSTNYH
SALISRVTISVDTSKNQFSLKLSSVTAADTAVYYCARPHYGSSYVGAMEYWGAGTTVT
VSS
Amino-acid Sequence of signal peptide underlined + AAS
(SEQ ID NO: 2)
METDTLLLWVLLLWVPGSTGAAS
Amino Acid Sequence of Construct #3 (without signaling peptide)
(SEQ ID NO: 20)
QVQLQESGPGLVKPSETLSLTCTVSGGSITSYGVSWIRQPPGKGLEWIGVIWEDGST
NYHSALISRVTISVDTSKNQFSLKLSSVTAADTAVYYCARPHYGSSYVGAMEYWGA
GTTVTVSSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDGGGGSGGG
GS

[symbol]

[symbol]

[symbol]

[symbol]

[symbol]

[symbol]

[symbol]

[symbol]

[symbol]

ASVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGECDKTHTCPP
CPAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALGAPIEKTISKAKGQPR
EPQVYTLPP [symbol] RDELTKNQVSL [symbol] CLVKGFYPSDIAVEWESNGQPENNYKTTPPVLDSDG
SFFLYSKLTVDKSRWQQGNVFSCSVMHEALHNHYTQKSLSLSPGK

### Construct #4, PLAP h4 VH-CH1—Fc(Hole) P329GLA-LA

Signaling peptide in italics, bold underlined+9 nucleotides cloning sites encoding AAS in italics regular font; PLAP h4 bold, CH1-underlined, Hole mutations shown in bold italics, larger font; 329GLA-LA bold underlined

Nucleotide sequence:
(SEQ ID NO: 21)

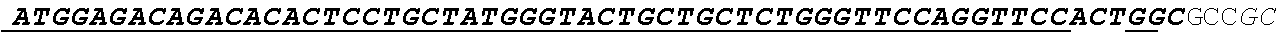

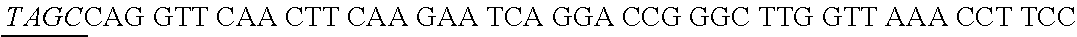

GAA ACT CTG AGC CTT ACT TGT ACAGTG TCT GGT GGA TCT ATT ACG AGC T
AC GGA GTA AGT TGG ATC CGG CAA CCA CCC GGG AAA GGG CTC GAATGG AT
A GGG GTG ATA TGG GAG GAT GGT TCA ACC AAC TAG CAT AGC GCT CTG AT
C AGC CGG GTG ACC ATTAGT GTC GAC ACT TCC AAA AAC CAG TTT TCA TTG
AAG CTC TCA AGC GTA ACT GCG GCG GAT ACC GCC GTATAC TAT TGT GCG
CGG CCA CAT TAC GGG TCC TCT TAT GTT GGG GCG ATG GAA TAT TGG GGG
GCA GGT ACAACG GTC ACG GTG TCT TCA

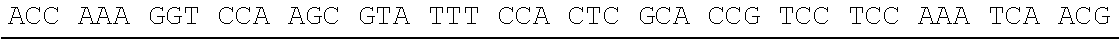

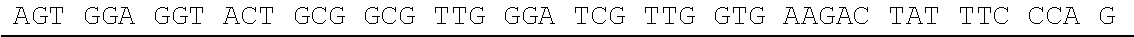

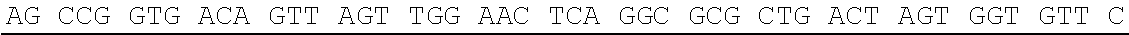

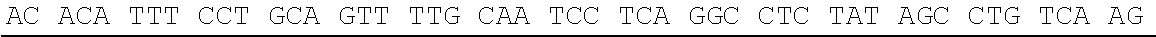

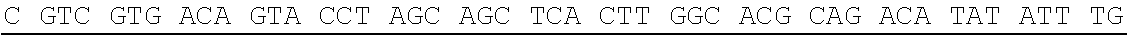

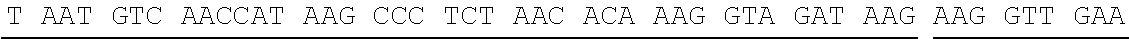

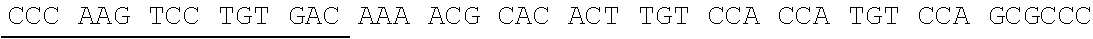

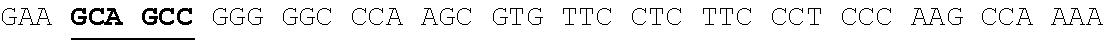

GAC ACC CTT ATG ATC TCA AGG ACT CCA GAA GTGACA TGC GTA GTC GTT G
AC GTA ACT CAC GAG GAT CCG GAA GTG AAG TTC AAC TGG TAG GTG GAC G
GT GTG GAG GTA CAT AAC GCGAAG ACT AAG CCC AGA GAA GAA CAA TAT AA
C TCA ACC TAG CGG GTC GTT TCT GTG CTC ACA GTG CTC CAC CAG GAC TG

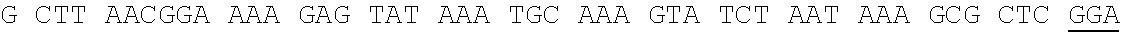

GCG CCC ATA GAG AAA ACT ATT TCT AAA GCA AAA GGT CAA CCACGG GAG

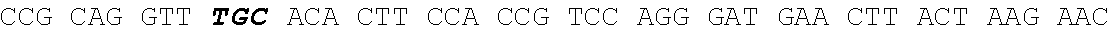

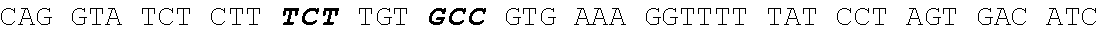

GCT GTC GAG TGG GAG AGO AAC GGT CAG CCG GAG AAT AAC TAT AAG ACC

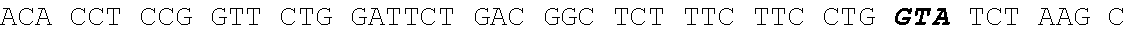

TT ACA GTC GAT AAA AG CGA TGG CAA CAA GGG AAT GTT TTT AGC TGC T
CT GTG . . . atgcat
GAGGCTCTGCACAACCACTACACGCAGAAGAGCCTCTCCCTGTCTCCCGGGAAATGA
Amino-acid of signaling peptide underlined + AAS
(SEQ ID NO: 2)
METDTLLLWVLLLWVPGSTGAAS
Amino-acid of Construct #4 (without signaling peptide)
(SEQ ID NO: 22)
QVQLQESGPGLVKPSETLSLTCTVSGGSITSYGVSWIRQPPGKGLEWIGVIWEDGST
NYHSALISRVTISVDTSKNQFSLKLSSVTAADTAVYYCARPHYGSSYVGAMEYWGA

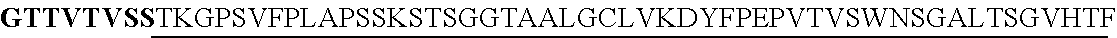

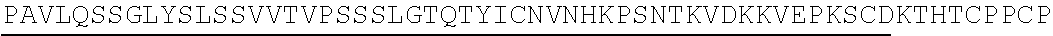

APEAAGGPSVFLFPPKPKDTLMISRTPEVTCVVVDVSHEDPEVKFNWYVDGVEVHNAK

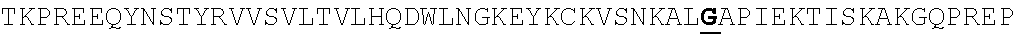

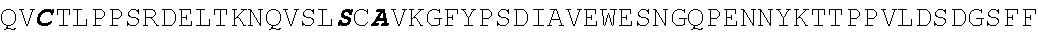

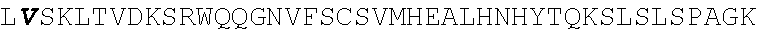

### Example 4

### The Sequence of PLAP H4-CD3 Antibody (FIG. 1B)

FIG. 1B shows the structure of humanized bivalent PLAP consisting of 3 DNA constructs. The structure has CD3 scFv (VH-linker-VL) attached to the C-terminal end of CH3. There is with no CROSS-Fab CD3.

PLAP h4-CD3e bivalent antibody (PBM0015) comprises 3 constructs:
1. PLAP h4 light chain, VL-CL: same as in example 3, construct #1.
2. PLAP h4 VH-CH1—Fc (knob) P329GLA-LA-CD3VH-linker-VL Amino acids of PLAP h4 VH-CH1, see Example 3, part of Construct 3.
3. PLAP h4VH-CH1—Fc (hole) same as construct #4 in example 3.

### Construct DNA#2

Construct #2: PLAP h4 VH-CH1—Fc (knob) P329GLA-LA-G4Sx3 linker-CD3VH-linker-VL

DNA was cloned to the same sites as in Example 3 to pYD11 vector.

### Nucleotide Sequence

Signaling peptide in italics bold, underlined+9 nucleotides cloning sites encoding AAS (italics, regular font); PLAPh4 VH (bold underlined); CH1 regular font, FC with (knob); P329GLA-LA mutations, regular font underlined; G4Sx2 linker bold, italics; CD3scFV (VH-G4Sx3-VL) is shown in bold italics, underlined

(SEQ ID NO: 23)

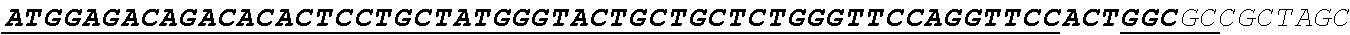

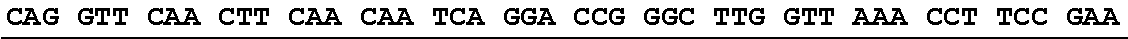

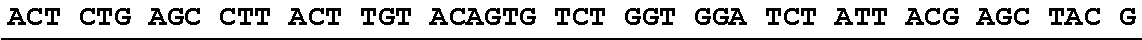

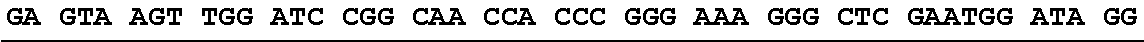

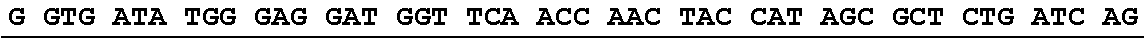

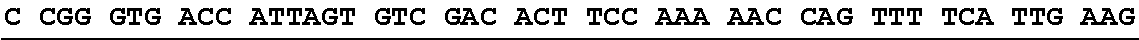

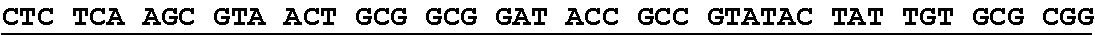

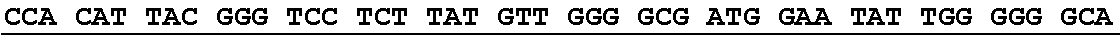

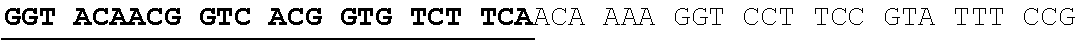

CTC GCA CCC AGC TCT AAG TCA ACC TCT GGC GGT ACT GCA GCC CTG GCT
TGC CTC GTA AAG GAC TAT TTT CCT GAG CCA GTA ACA GTT TCT TGG AAC
AGC GGG GCA CTT ACG AGC GGT GTT CAT ACG TTC CCT GCA GTG TTG CAA
TCC AGC GGC CTT TAT TCA TTG TCT TCA GTT GTA ACG GTT CCT TCT AGT
AGT TTG GGG ACC CAG ACA TAT ATC TGC AAC GTG AAC CAT AAG CCA AGC

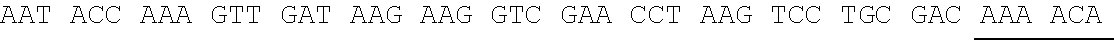

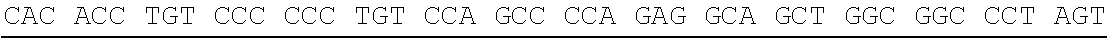

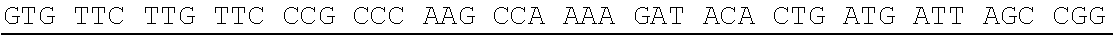

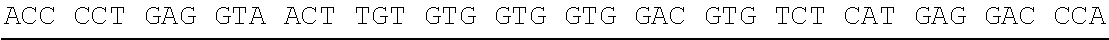

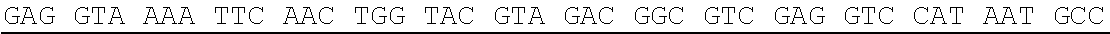

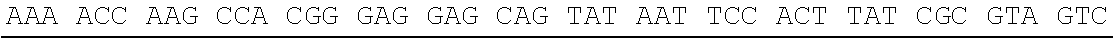

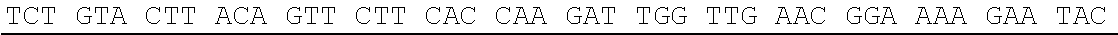

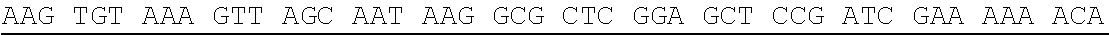

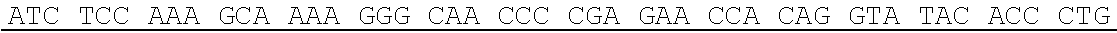

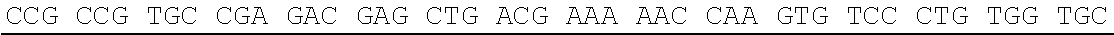

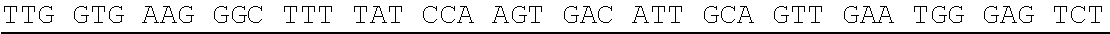

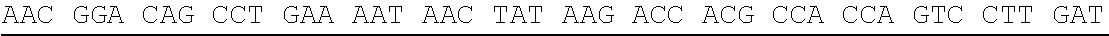

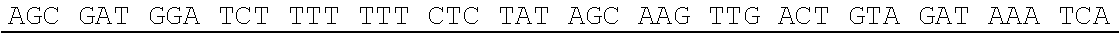

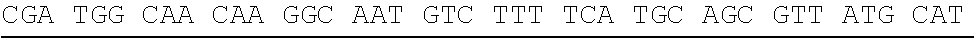

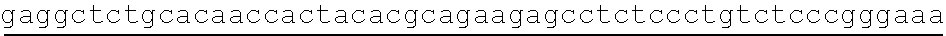

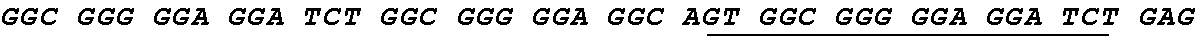

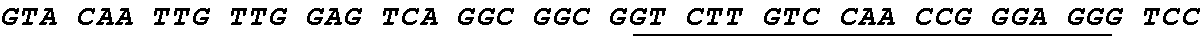

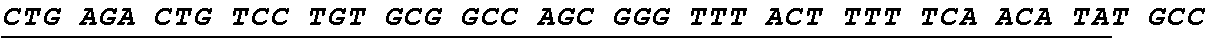

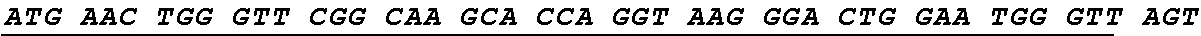

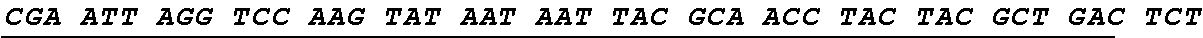

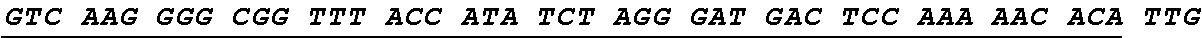

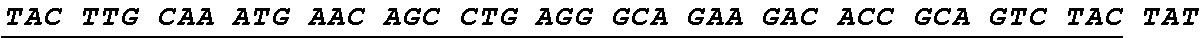

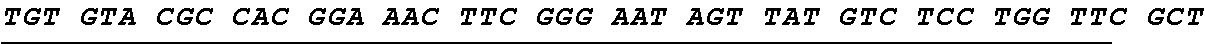

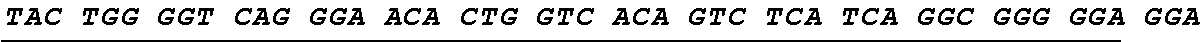

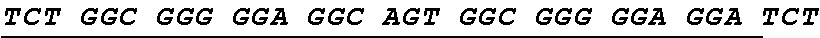

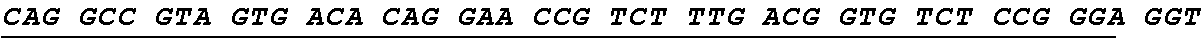

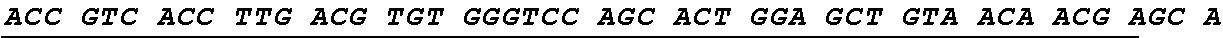

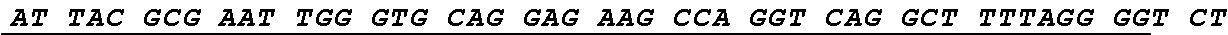

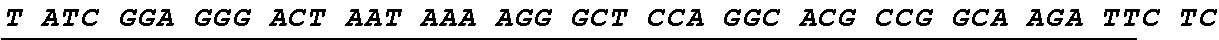

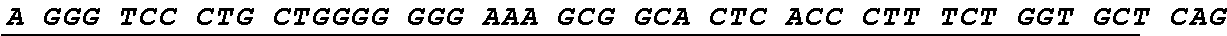

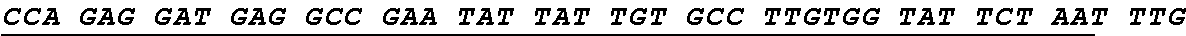

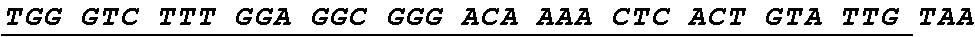

Construct 2, amino-acid without signaling peptide in front
(SEQ ID NO: 24)

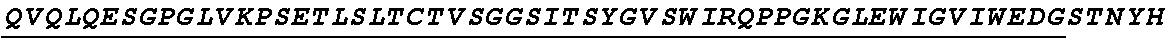

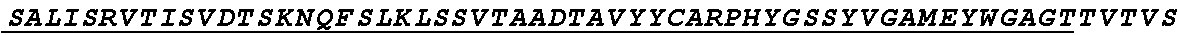

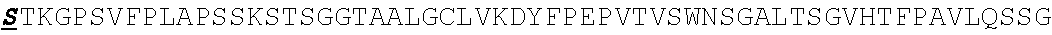

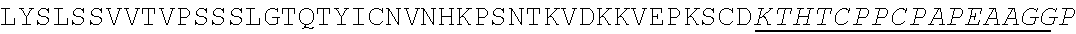

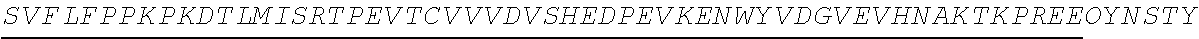

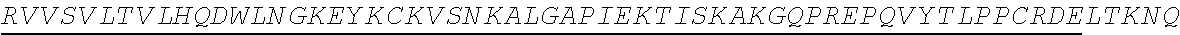

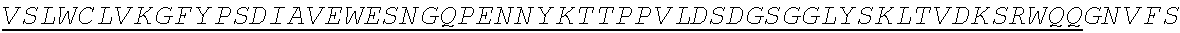

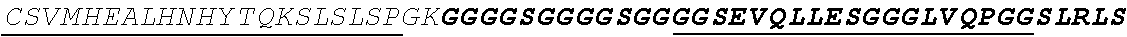

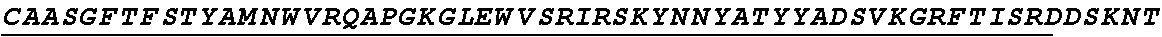

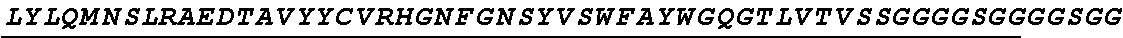

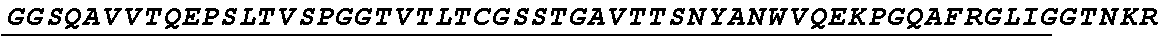

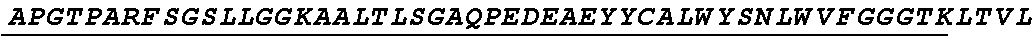

CD3 ScFV (VH underlined, linker italicized, VL, bold)
(SEQ ID NO: 25)

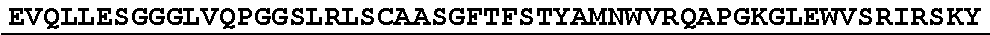

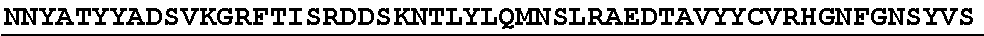

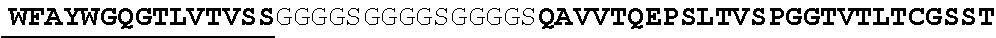

GAVTTSNYANWVQEKPGQAFRGLIGGTNKRAPGTPARFSGSLLGGKAALTLSGAQ
PEDEAEYYCALWYSNLWVFGGGTKLTVL

### Example 5

### The Sequence of PLAP H2-CD3 Antibody (FIG. 1C)

FIG. 1C shows the structure of monovalent humanized PLAP and monovalent CD3, which consists 3 DNA constructs. The structure does not have CD3 CROSSFAB, but is has CD3 scFv to bind CD3.

PLAP h2-CD3e monovalent antibody comprises 3 constructs:
Signaling peptide same as SEQ ID NO 2 except no AAS amino-acids at the end; METDTLLLWVLLLWVPGSTG (SEQ ID NO: 26).
1. PLAP h2 VL-CL, the amino-acid sequence is the same as that in EXAMPLE 2, Construct #1. The nucleotide sequence is different due to codon optimized.
2. PLAP h2 VH-CH1—Fc (knob)
3. CD3scFv-Fc (hole)

### Nucleotide Sequence of Construct 2

Signaling peptide underlined bold italics; PLAP h2 VH (bold)-CH1-Fc(knob): L234A; L235A mutations are shown in larger font underlined, bold, two knob mutations are in italics, larger font bold shown on FIG. 1C.

(SEQ ID NO: 27)

[symbol]

[symbol]

[symbol]

[symbol]

GAAGTGCAGCAGGTGCAGCTTCAGGAAAGTGGACCGGGCCTTGTCAAAC
CGTCAGAGACCCTTTCACTGACTTGCACTGTAAGTGGTTTCTCCCTGACAAGCT
ACGGAGTCTCCTGGATACGCCAGCCAGCGGGGAAAGGGCTTGAGTGGATCGGT
GTGATCTGGGAAGACGGGAGTACAAACTATCACTCAGCACTCATTAGTCGAGT
AACAATGTCCGTTGACACTTCCAAGAATCAATTCAGTTTGAAACTGTCTAGTGT
GACGGCTGCGGATACAGCGGTTTATTACTGTGCCAGGCCTCATTACGGAAGTT
CTTATGTTGGTGCAATGGAGTATTGGGGAGCCGGCACAACTGTCACTGTGAGC
TCCGTCACCGTCTCAAGCGCCTCCACCAAGGGCCCATCGGTCTTCCCGCTAGCACC
CTCCTCCAAGAGCACCTCTGGGGGCACAGCGGCCCTGGGCTGCCTGGTCAAGGACT
ACTTCCCCGAACCGGTGACGGTGTCGTGGAACTCAGGCGCCCTGACCAGCGGCGTC
CACACCTTCCCGGCTGTCCTACAGTCCTCCGGACTCTACTCCCTCAGCAGCGTAGTG
ACCGTGCCCTCCAGCAGCTTGGGCACCCAGACCTACATCTGCAACGTGAATCACAA
GCCCAGCAACACCAAGGTGGACAAGAAAGTTGAGCCCAAATCTTGTGACAAAACTC
ACACATGCCCACCGTGCCCAGCACCTGAA [symbol] GGGGGACCGTCAGTCTTCCT
CTTCCCCCCAAAACCCAAGGACACCCTCATGATCTCCCGGACCCCTGAGGTCACATG
CGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACAACAG
CACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCA
TCTCCAAAGCCAAAGGGCAGCCCCGAGAACCACAGGTGTACACCCTGCCCCCA [symbol]
CGGGATGAGCTGACCAAGAACCAGGTCAGCCTG [symbol] TGCCTGGTCAAAGGCTTCTA
TCCCAGCGACATCGCCGTGGAGTGGGAGAGCAATGGGCAGCCGGAGAACAACTACA
AGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTAAATG
A.

### Amino-Acid of Construct 2, PLAP h2 VH-CH1-Fc (Knob) (No Signaling Peptide)

PLAP h2 VH, underlined CH1; Fc in italics with mutations LA-LA in larger font and knock mutations underlined.

(SEQ ID NO: 28)

[symbol]

[symbol]

[symbol]

[symbol]

[symbol]

[symbol]

[symbol]

VTVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPE [symbol] GGPS
VFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPP [symbol] RDELTKNQV
SL [symbol] CLVKGFYPSDIAVEWESNGQPENNYKTTPPVLDSDGSFFLYSKLTVDKSRWQQGNVFSC
SVMHEALHNHYTQKSLSLSPGK

### Nucleotide Sequence of Construct 3, CD3scFv-Fc (Hole)

CD3 scFv in italics bold, then Fc (hole) with LA-LA mutations in bold underlined; hole mutations in italics larger font bold.

(SEQ ID NO: 29)
ATGGAGACAGACACACTCCTGCTATGGGTACTGCTGCTCTGGGTTCCAGGGTCGACT
GGC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GCGGCGGAGGAAGTGCGGCCGCG
ACTCACACATGCCCACCGTGCCCAGCACCTGAAGCCGCGGGGGGACCGTCAGTCTTCCTCTTCCCCC
CAAAACCCAAGGACACCCTCATGATCTCCCGGACCCCTGAGGTCACATGCGTGGTGGTGGACGTGAGC
CACGAAGACCCTGAGGTCAAGTTCAACTGGTACGTGGACGGCGTGGAGGTGCATAATGCCAAGACAA
AGCCGCGGGAGGAGCAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAGCCCCCATCGAGAAA
ACCATCTCCAAAGCCAAAGGGCAGCCCCGAGAACCACAGGTG [symbol] ACCCTGCCCCCATCCCGGGATG
AGCTGACCAAGAACCAGGTCAGCCTG [symbol] TGC [symbol] GTCAAAGGCTTCTATCCCAGCGACATCGCCGT
GGAGTGGGAGAGCAATGGGCAGCCGGAGAACAACTACAAGACCACGCCTCCCGTGCTGGACTCCGAC
GGCTCCTTCTTCCTC [symbol] AGCAAGCTCACCGTGGACAAGAGCAGGTGGCAGCAGGGGAACGTCTTCT
CATGCTCCGTGATGCATGAGGCTCTGCACAACCACTACACGCAGAAGAGCCTCTCCCTGTCTCCCGGG
AAATGA

### Amino-Acid of Construct 3, CD3scFv-Fc (Hole), Without Signaling Peptide

CD3 scFv in bold (linker underlined between CD3 VH and VL), in italics, FC in italics, L234A; L235A mutations in larger font; hole mutations (Y349C; T366S; L368A; Y407V underlined in bold, larger font as shown on FIG. 1C.

(SEQ ID NO: 30)

[symbol]

[symbol]

[symbol]

[symbol]

[symbol]

[symbol]

[symbol]

[symbol]

[symbol]

[symbol]

[symbol]

[symbol]

[symbol]

GGGGSAAATHTCPPCPAPEAAGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPAPIEKTISKAKGQPREPQV [symbol] TLPPSRDELTKNQVSL [symbol] C [symbol] VKGFYPSDIAVEWES
NGQPENNYKTTPPVLDSDGSFFL [symbol] SKLTVDKSRWQQGNVFSCSVMHEALHNHYTQKSLSLSP
GK

### Example 6

### Expression of PLAP H2 and PLAP H4-CD3 Antibodies (FIG. 1A)

293S cells were used that were grown in Freestyle F17 Expression serum free medium with 8 mM L-Glutamine (or GlutaMAX); 0.1% Pluoronic F-68. For transfection NanoFect Transfection Reagent was used at ratio 3:1 (3 microliters for 1 microg DNA). Harvest the supernatant after 3-7 days of transfection.

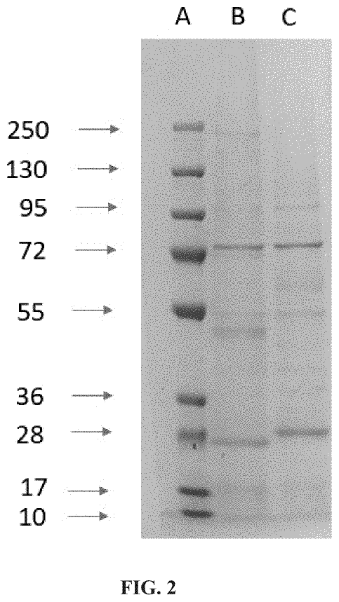

The antibody protein supernatants were expressed and run on the SDS gel at reduced and non-reduced condition (adding beta-mercaptoethanol to lysis buffer) (FIG. 2). The gel showed 4 bands.

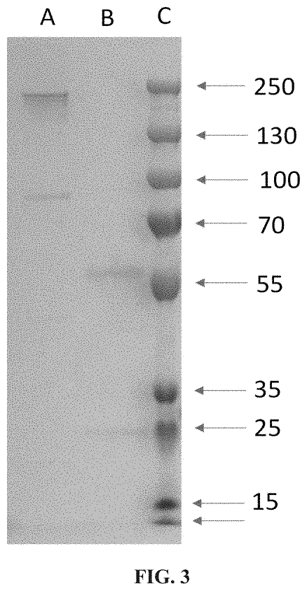

The protein was also purified using protein A or G columns. The purification was done with Millipore Sigma Protein A beads and Thermo IgG Elution buffer (Catalog number: 21004). After collection the samples were dialyzed using the Thermo Fisher Slide-A-Lyzer MINI Dialysis Devices. FIG. 3 shows purified PLAP h2-CD3 antibodies on SDS gel. The purified PLAP h2 antibody shows upper 206 kDA band at non-reducing conditions (A), this band disappears at reducing conditions (B).

### Example 7

### Binding of CD3 and PLAP Antigens by FACS

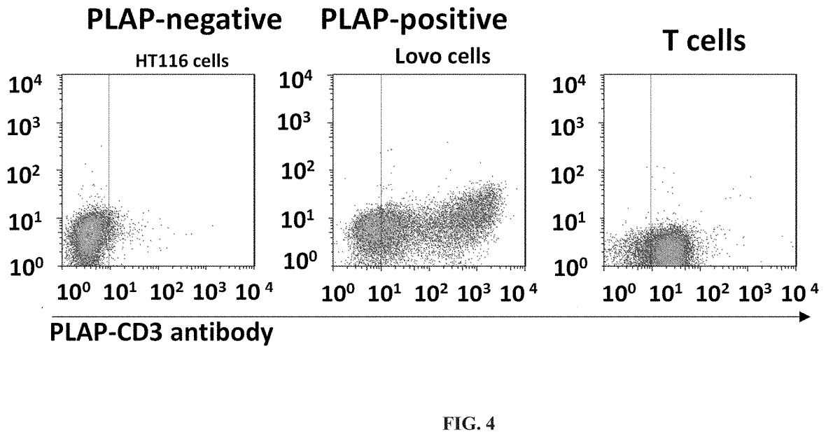

The FACS using bispecific PLAh2 and PLAP h4 antibodies (FIG. 1A) demonstrates that both antibodies bind to PLAP in PLAP-positive cells, and CD3 using T cells (FIG. 4).

The bispecific antibodies were tested with PLAP-positive and PLAP-negative cell lines. CD3-positive T cells were used for testing binding to CD3. Bispecific antibodies had positive binding with both PLAP and CD3 antigens. FIG. 4 shows the results of PLAP h2 -CD3 antibody. Similar result was observed for PLAP h4-Cd3 antibody (data not shown).

### Example 8

### Cytotoxic Activity of PLAP-CD3 Antibody With T Cells on PLAP-Positive Cell Target Line

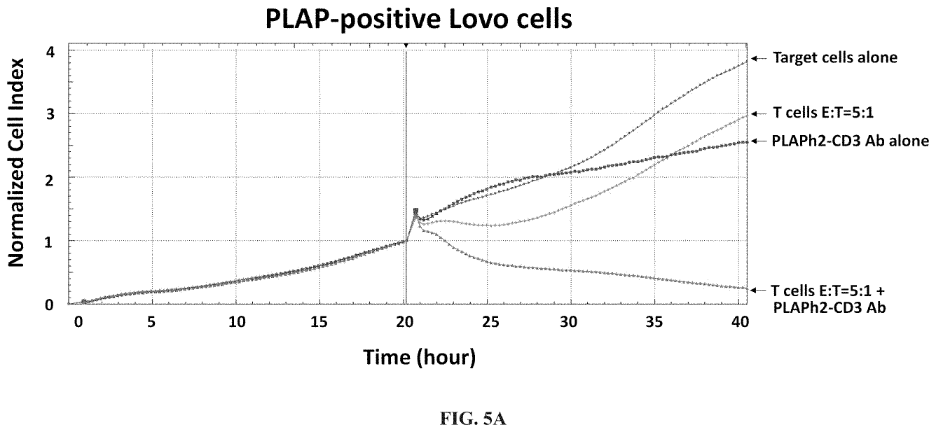

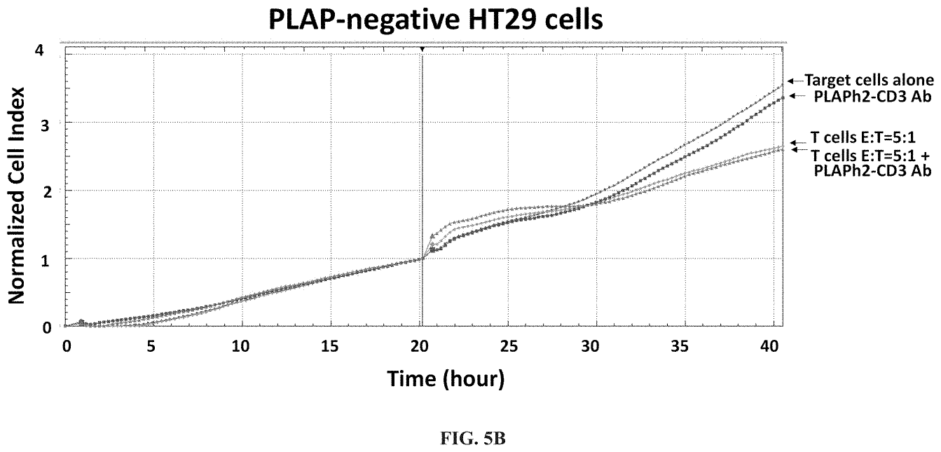

The antibody supernatants together with T cells were used for RTCA assay. Both bispecific antibodies added with activated T cells killed PLAP-positive cells and did not kill without T cells. PLAP-h2-CD3 plus T cells killed PLAP-positive cells and did not kill PLAP-negative HT29 cells (FIGS. 5A-5B). Antibody alone did not kill colon cancer cell line. T cells alone also did not kill target cells. This demonstrates high specificity of bispecific antibody when used together with T cells confirming mechanism of bringing T cells to cancer cells through bispecific antibody binding to CD3 antigen in T cells and to PLAP antigen.

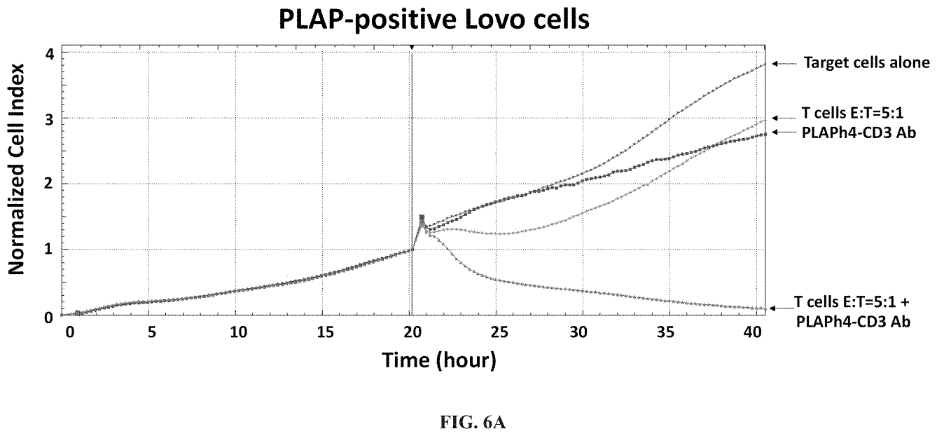

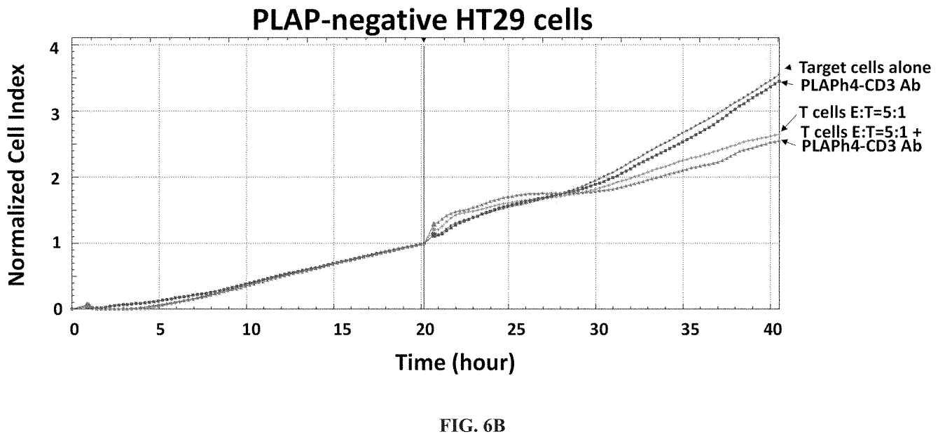

PLAP h4-CD3 antibody when used with activated T cells killed PLAP-positive cells and did not kill PLAP-negative cells (FIGS. 6A=6B). PLAP h4-CD3 antibody alone did not kill PLAP-positive target cells. In addition, bispecific antibodies demonstrated dose-dependent activity (not shown).

### Example 9

### In vivo Activity in Mice

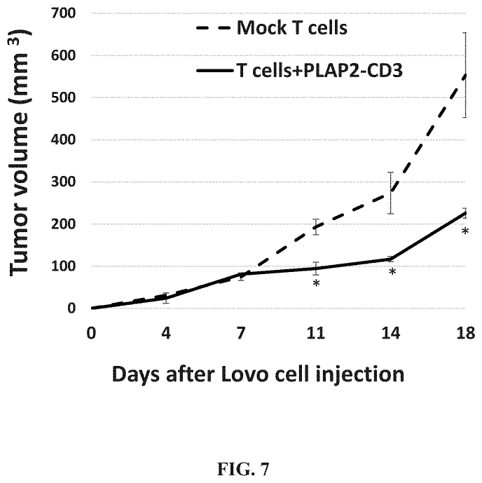

We administered bispecific antibody PLAP h2-CD3 (FIG. 1A structure) with T cells in Lovo xenograft mouse model (FIG. 7). The first injection of 1×10\hat{ }⁷ T cells was done at day 4, and bispecific antibody (10 micrograms to each mice or 0.5 mg/kg) was injected intravenously (iv) at day 7; then T cells with antibody were injected together by iv on days 7, 10, 14 and 17. Bispecific PLAP h2-CD3 antibody with T cells significantly decreased xenograft tumor growth (FIG. 7).

### Example 10

### Bivalent Humanized PLAP H4-CD3 SCFV Plus T Cells Specifically Killed PLAP-Positive Cells and Secrete IFN-Gamma

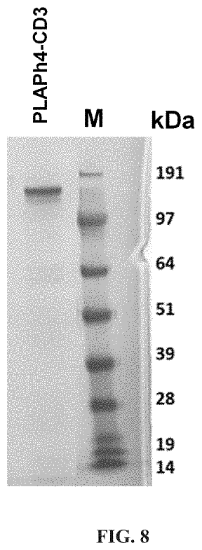

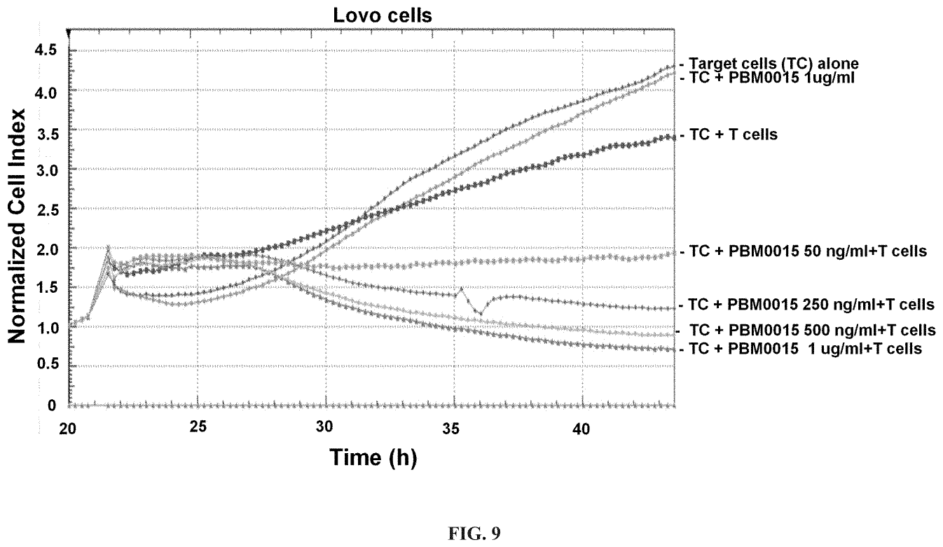

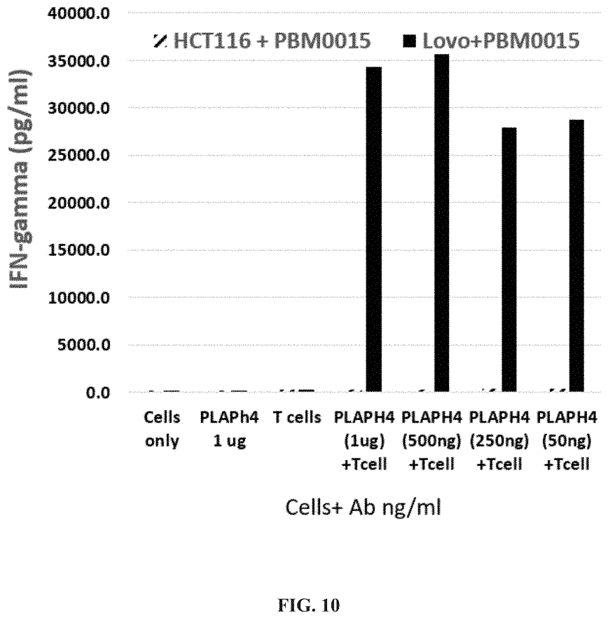

The bivalent bispecific humanized PLAPh4 with CD3 ScFv antibody (see FIG. 1B, PBM0015) showed as a single band on SDS gel (FIG. 8) with molecular weight around 130 kDa. PBM0015 antibody specifically bound to PLAP in Lovo cells and not to HCT116 (PLAP-negative cells); it also bound to CD3 as detected by FACS (not shown). PBM0015 antibody and T cells specifically killed PLAP-positive Lovo target cells in a dose-dependent manner (FIG. 9) and had minimal killing of PLAP-negative HCT116 cells (not shown). PBM0015 antibody with T cells secreted high level of IFN-gamma with Lovo cells but not with PLAP-negative HCT116 (FIG. 10). The results demonstrate high and specific activity of this antibody.

### Example 11

### Univalent PLAP H2-CD3 SCFV Antibody With T Cells Specifically Killed PLAP-Positive Cells and Secreted IFN-Gamma

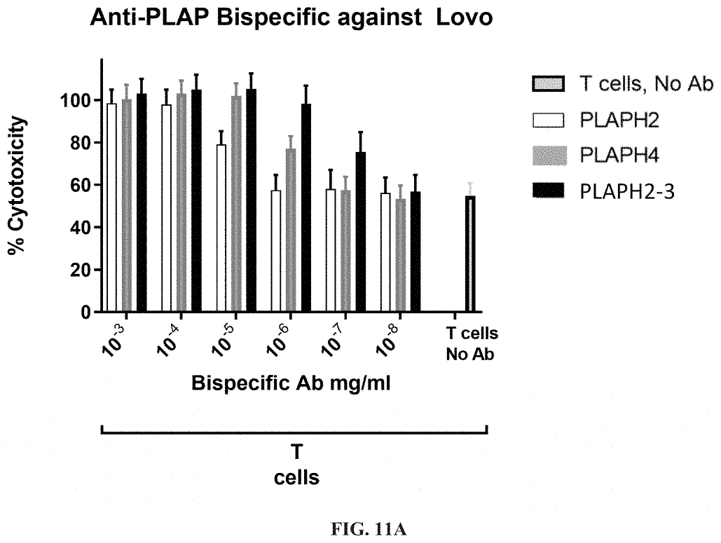

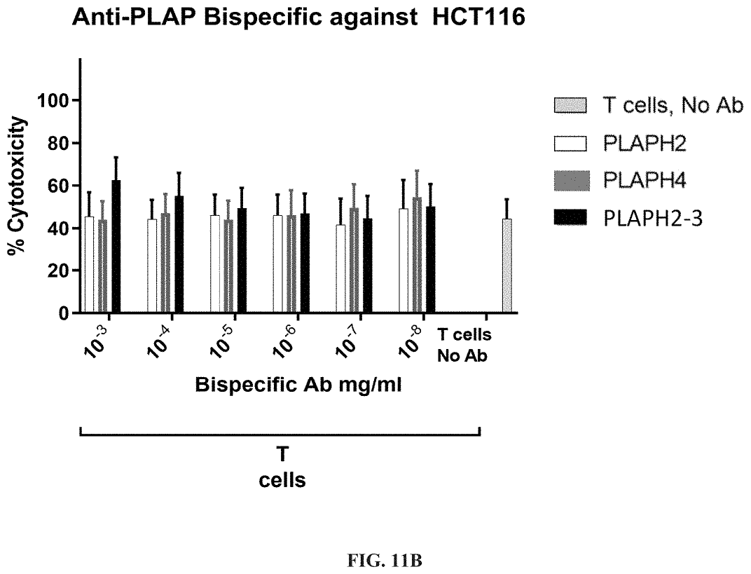

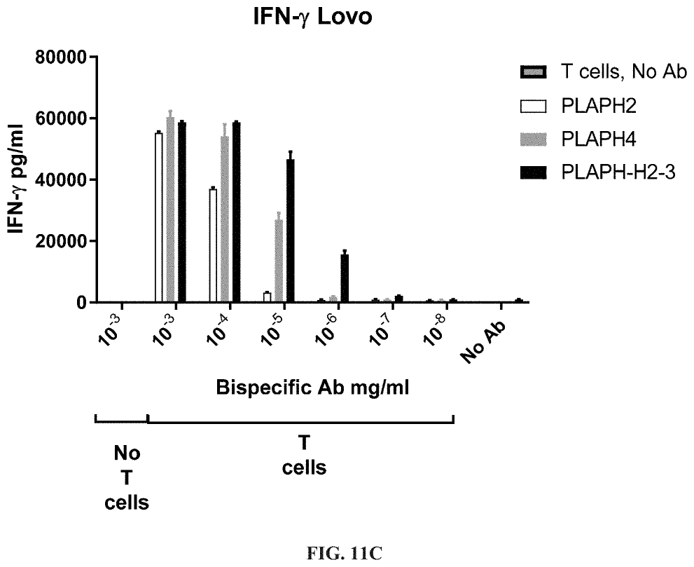

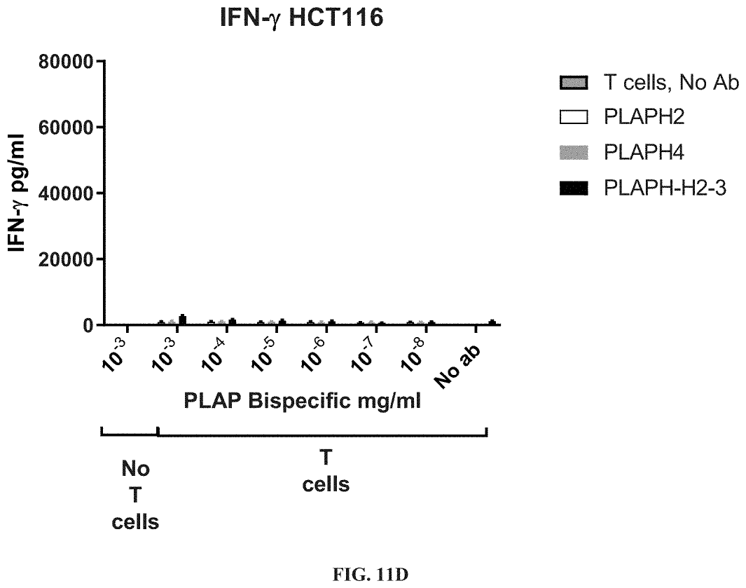

The bispecific univalent humanized PLAP h2 with CD3 Scfv antibody with structure as shown in FIG. 1C (PLAPh2-3) was run as one band on SDS gel (MW>100 kDa) (not shown). Humanized PLAP h2-CD3 antibody bound to PLAP in PLAP-positive Lovo, LS123 cells and not in HCT116 cells, it also bound to CD3 by FACS analysis (not shown). PLAPh2-3 antibody and T cells specifically killed PLAP-positive Lovo target cells and did not kill PLAP-negative cells (FIGS. 11A-B). The cytotoxic activity was similar or higher than PLAPh2 and PLAPh4 having the structure of FIG. 1A. The PLAPh2-3 Ab with T cells also secreted significant level of IFN-gamma with PLAP-positive cells but not with PLAP-negative cells (FIGS. 11C-D).

### REFERENCES

1. H. Durbin, D. F. Tucker, E. M. Milligan, L. G. Bobrow, P. H. Warne, Y. L. Pookim and W. F. Bodmer: Production of monoclonal antibodies to placental alkaline phosphatase: preliminary characterisation includes identification of one antibody reactive with routinely fixed histological preparations. Int J Cancer Suppl, 2, 50-8 (1988)

2. D. F. Tucker, R. T. Oliver, P. Travers and W. F. Bodmer: Serum marker potential of placental alkaline phosphatase-like activity in testicular germ cell tumours evaluated by H17E2 monoclonal antibody assay. Br J Cancer, 51 (5), 631-9 (1985)

3. A. A. Epenetos, A. J. Munro, D. F. Tucker, W. Gregory, W. Duncan, R. H. MacDougall, M. Faux, P. Travers and W. F. Bodmer: Monoclonal antibody assay of serum placental alkaline phosphatase in the monitoring of testicular tumours. Br J Cancer, 51 (5), 641-4 (1985)

4. M. Orsaria, A. P. Londero, S. Marzinotto, C. Di Loreto, D. Marchesoni and L. Mariuzzi: Placental type alkaline phosphatase tissue expression in ovarian serous carcinoma. Cancer Biomark, 17 (4), 479-486 (2016) doi:10.3233/CBM-160665

5. I. B. Vergote, V. M. Abeler, O. P. Bormer, T. Stigbrand, C. Trope and K. Nustad: CA125 and placental alkaline phosphatase as serum tumor markers in epithelial ovarian carcinoma. Tumour Biol, 13 (3), 168-74 (1992)

6. U. Harmenberg, M. Koha, R. Makiya, K. Koshida, B. Brismar, T. Stigbrand and B. Wahren: Identification and characterization of alkaline phosphatase isozymes in human colorectal adenocarcinomas. Tumour Biol, 12 (4), 237-48 (1991)

7. H. A. Muensch, W. C. Maslow, F. Azama, M. Bertrand, P. Dewhurst and B. Hartman: Placental-like alkaline phosphatase. Re-evaluation of the tumor marker with exclusion of smokers. Cancer, 58 (8), 1689-94 (1986)

8. B. Sandfeld-Paulsen, N. Aggerholm-Pedersen, R. Baek, K. R. Jakobsen, P. Meldgaard, B. H. Folkersen, T. R. Rasmussen, K. Varming, M. M. Jorgensen and B. S. Sorensen: Exosomal proteins as prognostic biomarkers in non-small cell lung cancer. Mol Oncol, 10 (10), 1595-1602 (2016) doi:10.1016/j.molonc.2016.10.003

9. R. Berahovich, H. Zhou, S. Xu, Y. Wei, J. Guan, J. Guan, H. Harto, S. Fu, K. Yang, S. Zhu, L. Li, L. Wu and V. Golubovskaya: CAR-T Cells Based on Novel BCMA Monoclonal Antibody Block Multiple Myeloma Cell Growth. Cancers (Basel), 10 (9) (2018) doi:10.3390/cancers10090323

10. M. Bacac, T. Fauti, J. Sam, S. Colombetti, T. Weinzierl, D. Ouaret, W. Bodmer, S. Lehmann, T. Hofer, R. J. Hosse, E. Moessner, O. Ast, P. Bruenker, S. Grau-Richards, T. Schaller, A. Seidl, C. Gerdes, M. Perro, V. Nicolini, N. Steinhoff, S. Dudal, S. Neumann, T. von Hirschheydt, C. Jaeger, J. Saro, V. Karanikas, C. Klein and P. Umana: A Novel Carcinoembryonic Antigen T-Cell Bispecific Antibody (CEA TCB) for the Treatment of Solid Tumors. Clin Cancer Res, 22 (13), 3286-97 (2016) doi:10.1158/1078-0432.CCR-15-1696

11. T. Schlothauer, S. Herter, C. F. Koller, S. Grau-Richards, V. Steinhart, C. Spick, M. Kubbies, C. Klein, P. Umana and E. Mossner: Novel human IgG1 and IgG4 Fc-engineered antibodies with completely abolished immune effector functions. Protein Eng Des Sel, 29 (10), 457-466 (2016) doi:10.1093/protein/gzw040

What is claimed is:

1. A bispecific antigen-binding molecule comprising: (a) a first and a second antigen-binding moiety each of which is a humanized Fab molecule capable of specific binding to human PLAP, and each comprises a heavy chain variable region (PALP VH) having the amino acid sequence of SEQ ID NO: 10 and a light chain variable region (PLAP VL) having the amino acid sequence of SEQ ID NO: 4, or each comprises a PLAP VH having the amino acid sequence of SEQ ID NO: 19 and a PLAP VL having the amino acid sequence of SEQ ID NO: 16; (b) a third antigen-binding moiety which is a Fab molecule capable of specific binding to human CD3 epsilon, the third antigen-binding moiety comprises a heavy chain variable region (CD3 VH) having the amino acid sequence of SEQ ID NO: 11 and a light chain variable region (CD3 VL) having the amino acid sequence of SEQ ID NO: 7, wherein the third antigen-binding moiety is a crossover Fab molecule, in which the constant regions of the Fab light chain and the Fab heavy chain are exchanged; and (c) an human IgG Fc domain comprising a first subunit and a second subunit capable of stable association;
wherein the Fab heavy chain of the third antigen-binding moiety is (i) fused at the N-terminus to the C-terminus of the Fab heavy chain of the first antigen-binding moiety (CH1), and (ii) fused at the C-terminus to the N-terminus of the first subunit of the Fc knob domain, and wherein the second antigen-binding moiety is fused at the C-terminus of the Fab heavy chain (CH1) to the N-terminus of the second subunit of the Fc hole domain.

2. The bispecific antigen-binding molecule of claim 1, wherein the PLAP VH comprises the amino acid sequence of SEQ ID NO: 10 and the PLAP VL comprises the amino acid sequence of SEQ ID NO: 4.

3. The bispecific antigen-binding molecule of claim 2, wherein the human IgG Fc domain comprises one or more amino acid substitutions promoting the association of the first and the second subunit of the Fc domain.

4. The bispecific antigen-binding molecule of claim 3, wherein said one or more amino acid substitutions are at one or more positions selected from the group of L234, L235, and P329, according to EU numbering.

5. The bispecific antigen-binding molecule of claim 2, wherein one of the subunits of the human IgG Fc domain comprises mutations of S354C and T366W, and the other one of the subunits of the human Fc domain comprises mutations of Y349C, T366S, L368A and Y407V, according to EU numbering.

6. The bispecific antigen-binding molecule of claim 4, wherein one of the subunits of the human IgG Fc domain comprises mutations of S354C and T366W, and the other one of the subunits of the human Fc domain comprises mutations of Y349C, T366S, L368A and Y407V, according to EU numbering.

7. The bispecific antigen-binding molecule of claim 2, comprising the amino acid sequences of SEQ ID NO: 5, 8, 12, and 14, in a molar ratio of 2:1:1:1.

8. The bispecific antigen-binding molecule of claim 1, wherein the PLAP VH comprises the amino acid sequence of SEQ ID NO: 19 and the PLAP VL comprises the amino acid sequence of SEQ ID NO: 16.

9. The bispecific antigen-binding molecule of claim 8, wherein the human IgG Fc domain comprises one or more amino acid substitutions promoting the association of the first and the second subunit of the Fc domain.

10. The bispecific antigen-binding molecule of claim 9, wherein said one or more amino acid substitutions are at one or more positions selected from the group of L234, L235, and P329, according to EU numbering.

11. The bispecific antigen-binding molecule of claim 8, wherein one of the subunits of the Fc domain comprises mutations of S354C and T366W, and the other one of the subunits of the Fc domain comprises mutations of Y349C, T366S, L368A and Y407V, according to EU numbering.

12. The bispecific antigen-binding molecule of claim 10, wherein one of the subunits of the Fc domain comprises mutations of S354C and T366W, and the other one of the subunits of the Fc domain comprises mutations of Y349C, T366S, L368A and Y407V, according to EU numbering.

13. The bispecific antigen-binding molecule of claim 8, comprising the amino acid sequences of SEQ ID NO: 17, 8, 20, and 22, or at least 95% sequence identity thereof, in a molar ratio of 2:1:1:1.

14. A bispecific antigen-binding molecule comprising two binding moieties to PLAP, and one binding moiety to CD3 epsilon, the molecule comprises the amino acid sequences of SEQ ID NO: 17, 24, and 22, or at least 95% sequence identity thereof, in a molar ratio of 2:1:1.

15. A bispecific antigen-binding molecule comprising one binding moiety to PLAP, and one binding moiety to CD3 epsilon, wherein the molecule comprises the amino acid sequences of SEQ ID NO: 5, 28, and 30, or the amino acid sequences of SEQ ID NO: 17, 28, and 30, or at least 95% sequence identity thereof, in a molar ratio of 2:1:1.
